SEQUENCE LISTING (7 sequences)

SEQ ID NO: 1 - DNA, 1119 bp, Ephedra sinica
atgagaggatcgcatcaccatcaccatcacggatccatggaagaagcaaaaatggcgaccctgggcggtgcgtcctatgcgatgattgtgaaaacgatgatgcgctctctggaagcaaacctgattccggattttgtgctgcgtcgcctgacgcgtatcctgctggctagtcgcctgaaactgggttataagcagaccgctgaactgcaactggcggatctgatgtcattcgttgcgtcgctgaaaacgatgccgattgccctgtgcaccgaagaagcaaagggtcagcattacgaactgccgaccagctttttcaaactggtcctgggcaaacatctgaagtatagctctgcctacttttctgaacacacccgtacgctggatgaagcggaagaagccatgctggcactgtattgcgaacgcgccaaaattgaagatggtcagaagattctggacatcggctgtggttggggcagtttttccctgtatgtggcagaacgttacccgaaatgcgaaattacgggcctgtgtaacagttccacccaaaaagccttcatcgaacagcaatgcagcgaacgtcgcctgtgtaatgttaccatttatgcagatgacatcagcacctttgatacggaatctacctacgaccgcattatcagcatcgaaatgttcgaacacatgaagaactacagtacgctgctgaagaaaattagcaagtggatgaatcaggaatgcctgctgtttgtccattatttctgtcacaaaacctttgcgtaccacttcgaagatgtggacgaagatgactggatggctcgttatttctttaccggcggcaccatgccggcgtcatcgctgctgctgtactttcaggatgacgtctcagtggttgatcattggctgattaacggtaaacactatgctcaaacctcggaagaatggctgaagcgtatggaccacaatctgagctctattctgccgatctttaacgaaacgtatggcgaaaatgcggccaaaaagtggctggcatactggcgcacctttttcatcgcagttgctgaactgttcaaatacaacgatggcgaagaatggatggtgtcccacttcctgttcaaaaagaaataa

SEQ ID NO: 2 - protein, 372 aa, Ephedra sinica
MRGSHHHHHHGSMEEAKMATLGGASYAMIVKTMMRSLEANLIPDFVLRRLTRILLASRLKLGYKQTAELQLADLMSFVASLKTMPIALCTEEAKGQHYELPTSFFKLVLGKHLKYSSAYFSEHTRTLDEAEEAMLALYCERAKIEDGQKILDIGCGWGSFSLYVAERYPKCEITGLCNSSTQKAFIEQQCSERRLCNVTIYADDISTFDTESTYDRIISIEMFEHMKNYSTLLKKISKWMNQECLLFVHYFCHKTFAYHFEDVDEDDWMARYFFTGGTMPASSLLLYFQDDVSVVDHWLINGKHYAQTSEEWLKRMDHNLSSILPIFNETYGENAAKKWLAYWRTFFIAVAELFKYNDGEEWMVSHFLFKKK

SEQ ID NO: 3 - protein, 388 aa, Glaucium flavum
MGSSHHHHHHGTGSYITSLYKKAGWDLMATSKQVKKKEELLKNMELGLVPDEEIRRLIRIELEKRLKWGYKPTHQQQLAQLLDLVHSLKKMKIATEMESLDLKLYEAPFSFVQIKHGSTIKESSSYFKDESMTLDEAEIAMLDLYVERAQIEDGQSVLDLGCGLGAVTLHVAKKYKNCHVTGLTNSVEQKDFIEGKCKELNLSNVKVILADVTSHEMEDKFDRIFAVELIEHMKNYELLLRRISKWMKDDGLLFIEHVCHKTFAYHYEPIDEDDWYTEYIFPAGTLTLSSASLLLYFQDDVSVVNHWTLSGKHYSRSHEEWLKRIDGNMDAVKEIMKSITKTEEEAVKLINFWRIFCMCGAELFGYKDGEEWMMSHVLFKKKQLLQQC

SEQ ID NO: 4 - protein, 382 aa, Glaucium flavum
MGSSHHHHHHGTGSYITSLYKKAGWGSNEAQVKKESIGEIMGKLMQGEIGDEELSKRIKEIFGKRLQWGYKPTHQQQLAFNLDFIKSLKEMDMSGEIDTMNEETYELPSAFLEAAFGKTIKQSGCYFKDETTTIDEAEEASHELYCERAQIKDGQTVLDIGCGQGGLVLHIAQKYKNCHVTGLTNSKAQKNYILMQAEKLQLSNVDVILADVTKHESDKTYDRILVIETIEHMKNIQLFMKKLSTWMTEDSLLFVDHICHKTFSHHFEAIDEDDWYSGFIFPKGCVTILSASALLYFQDDVTILDHWVVNGMHMARSVDAWRKKLDKNMELAREILLPGLGSKEAVNGVITHIRTFCMGGYEQFSYNNGEEWMVAQMLFKKK

SEQ ID NO: 5 - protein, 387 aa, Papaver somniferum
MGSSHHHHHHGTGSYITSLYKKAGWSTTMETTKISQQDDLWKNMELGQISDEEVRRLMKIGIEKRIKWGTKPTQQEQLAQLLDFNKSLRGMKMATEIDTLENHKIYETPESFNQIIGGKESAGLFTDETTTTMEEANTKMMDLYCERAGLKDGHTILDLGCGAGLLVLHLAKKYKKSKITGITNTSSHKEYILKQCKNLNLSNVEIILADVTKVDIESTFDRVFVIGLIEHMKNFELFLRKISKWMKDDGLLLLEHLCHKSFSDHWEPLSEDDWYAKNFFPSGTLVIPSATCLLYFQEDVTVIDHWILSGNNFARSNEVILKRIDGKIEEVKDIFMSFYGIGREEAVKLINWWRLLCITANELFKYNNGEEWLISQLLFKKKLMTCI

SEQ ID NO: 6 - DNA, 1083 bp, Native Ephedra sinica
atggaagaggcgaagatggcaacgctgggcggtgcctcgtatgccatgatagtgaagacaatgatgcgctctctcgaagccaacctaattccggacttcgtactgagaaggctcactcgcattctcttggcgagtcgtctgaagctcggatacaagcaaaccgcggagcttcagcttgctgatctcatgtcctttgttgcatcgttaaagaccatgcctatagctctctgcaccgaagaagctaaaggccagcattacgagctgcccacttcgttcttcaagttggttctcggcaagcatctcaaatacagctcggcttacttcagtgaacacacaagaactctagatgaagcagaggaagcaatgttagcactgtattgtgagagggctaagattgaagatggccaaaagattctggatataggatgtggatggggttcattttctttatatgttgctgaaagatacccaaaatgtgaaataactggtctttgcaattcctctacccagaaggcgtttatagagcaacagtgcagtgaaagaagactttgcaatgttactatatatgctgatgatattagcacttttgatacggagagtacttatgatcgcataatatctattgaaatgtttgagcacatgaaaaattacagtactcttcttaaaaaaatatcaaagtggatgaatcaggaatgcttgctgtttgtgcattatttttgtcataagacatttgcttaccattttgaggatgtagatgaagatgattggatggcaagatactttttcacaggtggcacaatgcctgcaagtagcttactactgtattttcaggatgatgtttcagttgttgatcactggcttataaatgggaagcactatgctcaaacaagtgaagagtggctgaaaaggatggaccataatctatcctctatacttcctatatttaatgaaacatatggcgaaaatgcagctaaaaagtggttggcatactggcgaacattctttattgccgtggctgagctttttaagtacaatgatggagaagaatggatggtttcacattttctgtttaagaaaaaatag

SEQ ID NO: 7 - protein, 360 aa, Native Ephedra sinica
MEEAKMATLGGASYAMIVKTMMRSLEANLIPDFVLRRLTRILLASRLKLGYKQTAELQLADLMSFVASLKTMPIALCTEEAKGQHYELPTSFFKLVLGKHLKYSSAYFSEHTRTLDEAEEAMLALYCERAKIEDGQKILDIGCGWGSFSLYVAERYPKCEITGLCNSSTQKAFIEQQCSERRLCNVTIYADDISTFDTESTYDRIISIEMFEHMKNYSTLLKKISKWMNQECLLFVHYFCHKTFAYHFEDVDEDDWMARYFFTGGTMPASSLLLYFQDDVSVVDHWLINGKHYAQTSEEWLKRMDHNLSSILPIFNETYGENAAKKWLAYWRTFFIAVAELFKYNDGEEWMVSHFLFKKK

(19) United States
(12) Patent Application Publication
(10) Pub. No.: US 2021/0087593 A1
(43) Pub. Date: Mar. 25, 2021

(54) N-ALKYLATION OF ALKALOID COMPOUNDS

(71) Applicant: Willow BioSciences Inc., Calgary (CA)

(72) Inventors: Peter J. Facchini, Calgary (CA); Jillian M. Hagel, Calgary (CA); Jeremy S. Morris, Calgary (CA)

(21) Appl. No.: 16/635,224

(22) PCT Filed: Jul. 31, 2018
(86) PCT No.: PCT/CA2018/050932

§ 371 Date: Jan. 30, 2020

Related U.S. Application Data
(60) Provisional application No. 62/538,918, filed on Jul. 31, 2017.

(51) Int. Cl. C12P 13/00; C12P 17/12; C12P 17/10; C12P 17/18; C12N 9/10

(52) U.S. Cl. CPC C12P 13/001; C12P 17/12; C12Y 201/01049; C12P 17/182; C12N 9/1007; C12P 17/10

(57) ABSTRACT

Disclosed are methods for converting an alkaloid substrate compound into a N-alkylated alkaloid product compound in the presence of an alkyl donor compound and catalytic quantities of an N-alkyltransferase enzyme capable of converting the alkaloid substrate into an alkylated product. The N-alkyltransferase is obtainable from Ephedra sinica.

### RELATED APPLICATION

This Patent Cooperation Treaty Application claims the benefit under 35 USC § 119 (e) from U.S. Provisional Patent Application No. 62/538,918, filed on Jul. 31, 2017, which is incorporated by reference herein in its entirety.

### FIELD OF THE DISCLOSURE

The methods and systems disclosed herein relate to a class of chemical compounds known as alkaloids and methods of making alkaloids. In particular, the methods and systems disclosed herein relate to enzymes known as N-alkyltransferases for making alkaloid compounds.

### BACKGROUND OF THE DISCLOSURE

The following paragraphs are provided by way of background to the present disclosure. They are not however an admission that anything discussed therein is prior art or part of the knowledge of persons skilled in the art.

Alkaloids are a class of nitrogen containing organic chemical compounds that are naturally produced by opium poppy (Papaver somniferum), and a range of other plant species belonging to the Papaveraceae family of plants, as well as other plant families including, for example the Lauraceae, Annonaceae, Euphorbiaceae and the Moraceae. The interest of the art in alkaloid compounds is well established and can be explained by the pharmacological properties of these compounds, as well as their utility as feedstock materials in the manufacture of pharmaceutical compounds. Thus, alkaloids, such as tyrosine, coclaurine and reticuline can be used as a feedstock compounds to manufacture, for example, codeine and morphine.

In biosynthetic production systems for alkaloids, many substrate alkaloid compounds are not efficiently enzymatically converted into the desired products, for example, due to substrate inhibition, or they are converted into products other than the desired alkaloids products, each of which results into low alkaloid product yields. Thus, for example, in instances in which it is desired that in a substrate alkaloid, the nitrogen atom in the alkaloid is enzymatically alkylated to form an N-alkylated product alkaloid, the reaction can be inefficient. There exists therefore a need in the art for improved processes to obtain alkaloid synthesis enzymes and alkaloids, and in particular N-alkylated alkaloid compounds.

### SUMMARY OF THE DISCLOSURE

The following paragraphs are intended to introduce the reader to the more detailed description, not to define or limit the claimed subject matter of the present disclosure.

In one aspect, the present disclosure relates to alkaloid compounds.

In another aspect, the present disclosure relates to N-alkyltransferase enzymes useful in the synthesis of alkaloid compounds.

Accordingly, in one aspect, the present disclosure provides, in at least one embodiment, a method of making an alkaloid comprising:
(a) providing an alkaloid substrate selected from the group of substrates consisting of:
   (i) a first alkaloid compound (I):

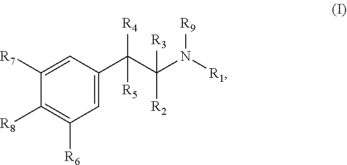

wherein
   R₁ is a hydrogen atom or an alkyl group;
   R₂ and R₃ are independently or simultaneously a hydrogen atom, a halogen or an alkyl group;
   R₄ is a hydrogen atom, a hydroxy group, a halogen, or an alkyl group;
   R₅ is a hydrogen atom, a halogen, or an alkyl group; or R₄ and R₅, taken together, form a carbonyl group;
   R₆ is a hydrogen atom, a halogen, an alkyl group or an alkoxy group;
   R₇ is a hydrogen atom, a halogen, an alkyl group or an alkoxy group;
   R₈ is a hydrogen atom, a hydroxy group, a halogen, an alkyl group or an alkoxy group;
   R₇ and R₈, taken together, form a methylenedioxy group; and
   R₉ is a hydrogen atom or an alkyl group;
(ii) a second alkaloid compound (II):

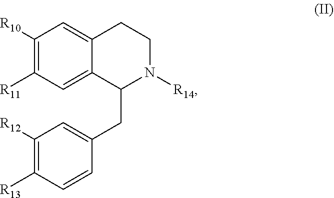

wherein
   R₁₀ is a hydrogen atom, a hydroxy group, a halogen, an alkyl group or an alkoxy group;
   R₁₁ is a hydrogen atom, a hydroxy group, a halogen, an alkyl group or an alkoxy group;
   R₁₂ is a hydrogen atom, a hydroxy group, a halogen, an alkyl group or an alkoxy group;
   R₁₃ is a hydrogen atom, a hydroxy group, a halogen, an alkyl group or an alkoxy group; and
   wherein R₁₀-R₁₃ are not each simultaneously a hydroxy group; and
   R₁₄ is a hydrogen atom or an alkyl group;
(iii) a third alkaloid compound (III):

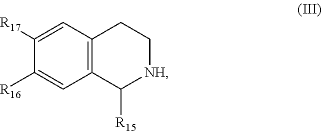

wherein
   R₁₅, R₁₆ and R₁₇ are independently or simultaneously a hydrogen atom, a hydroxy group, an alkoxy group, a halogen or an alkyl group; and
(iv) a fourth alkaloid compound (IV) selected from stylopine; tryptamine; harmaline; and propanolol; and
(b) contacting the alkaloid substrate with sufficient quantities of an alkyl donor compound and catalytic quantities of an enzyme encoded by a nucleic acid sequence selected from the group consisting of:
   (i) SEQ. ID NO: 1 or SEQ. ID NO: 6;
   (ii) a nucleic acid sequence that is substantially identical to SEQ. ID NO: 1 or SEQ. ID NO: 6;
   (iii) a nucleic acid sequence that is substantially identical to SEQ. ID NO: 1 or SEQ. ID NO: 6 but for the degeneration of the genetic code;
   (iv) a nucleic acid sequence that is complementary to SEQ. ID NO: 1 or SEQ. ID NO: 6;
   (v) a nucleic acid sequence encoding a polypeptide having the amino acid sequence set forth in SEQ. ID NO: 2 or SEQ. ID NO: 7;
   (vi) a nucleic acid sequence that encodes a functional variant of a polypeptide having the amino acid sequence set forth in SEQ. ID NO: 2 or SEQ. ID NO: 7; and
   (vii) a nucleic acid sequence that hybridizes under stringent conditions to any one of the nucleic acid sequences set forth in (i), (ii), (iii), (iv), (v) or (vi),
   under reaction conditions permitting an enzyme-catalyzed N-alkylation of the alkaloid substrate to form a N-alkylated alkaloid product.

In some embodiments, the alkyl donor compound can be a methyl donor compound and the enzyme can be a methyltransferase capable of N-methylation of the alkaloid substrate to form a N-methylated alkaloid product.

In some embodiments, R₁ can be a hydrogen atom or a (C₁-C₆)-alkyl group.

In some embodiments, R₁ can be a hydrogen atom, a methyl group, an ethyl group, a propyl group, a butyl group, or a pentyl group.

In some embodiments, R₂ can be a hydrogen atom, a (C₁-C₆)-alkyl group or a halogen.

In some embodiments, R₂ can be a hydrogen atom, a methyl group, an ethyl group, a propyl group, a butyl group, a pentyl group, fluorine, chlorine, bromine, or iodine.

In some embodiments, R₃ can be a hydrogen atom, a (C₁-C₆)-alkyl group or a halogen.

In some embodiments, R₃ can be a hydrogen atom, a methyl group, an ethyl group, a propyl group, a butyl group, a pentyl group, fluorine, chlorine, bromine, or iodine.

In some embodiments, R₄ can be a hydrogen atom, a hydroxy group, a (C₁-C₆)-alkyl group or a halogen.

In some embodiments, R₄ can be a hydrogen atom, a methyl group, an ethyl group, a propyl group, a butyl group, a pentyl group, fluorine, chlorine, bromine, or iodine.

In some embodiments, R₅ can be a hydrogen atom, a (C₁-C₆)-alkyl group or a halogen.

In some embodiments, R₅ can be a hydrogen atom, a methyl group, an ethyl group, a propyl group, a butyl group, a pentyl group, fluorine, chlorine, bromine, or iodine.

In some embodiments, R₄ can be a hydrogen atom or a hydroxy group, and R₅ can be a hydrogen atom.

In some embodiments, R₄ and R₅, taken together, can be a carbonyl group.

In some embodiments, R₆ can be a hydrogen atom, a (C₁-C₆)-alkoxy group, a (C₁-C₆)-alkyl group or a halogen.

In some embodiments, R₆ can be a hydrogen atom, a methyl group, an ethyl group, a propyl group, a butyl group, a pentyl group, a methoxy group, an ethoxy group, a propoxy group, a butoxy group, a pentoxy group, fluorine, chlorine, bromine, or iodine.

In some embodiments, R₇ can be a hydrogen atom, a (C₁-C₆)-alkoxy group, a (C₁-C₆)-alkyl group or a halogen.

In some embodiments, R₇ can be a hydrogen atom, a methyl group, an ethyl group, a propyl group, a butyl group, a pentyl group, a methoxy group, an ethoxy group, a propoxy group, a butoxy group, a pentoxy group, fluorine, chlorine, bromine, or iodine.

In some embodiments, Ra can be a hydrogen atom, a hydroxy group, a (C₁-C₆)-alkoxy group, a (C₁-C₆)-alkyl group or a halogen.

In some embodiments, R₈ can be a hydrogen atom, a hydroxy group, a methyl group, an ethyl group, a propyl group, a butyl group, a pentyl group, a methoxy group, an ethoxy group, a propoxy group, a butoxy group, a pentoxy group, fluorine, chlorine, bromine, or iodine.

In some embodiments, R₇ and R₈, taken together, can form a methylenedioxy group.

In some embodiments, R₉ can be a hydrogen atom or a (C₁-C₆)-alkyl group.

In some embodiments, R₉ can be a hydrogen atom, a methyl group, an ethyl group, a propyl group, a butyl group, or a pentyl group.

In some embodiments, R₁, R₄, R₈ and R₉ can not simultaneously be: a hydrogen atom, a hydroxy group, a hydroxy group and a hydrogen atom, respectively. In one embodiment, the first alkaloid compound (I) is not octopamine.

In some embodiments, R₁, R₄, R₈ and R₉ can not simultaneously be: a hydrogen atom, a hydroxy group, a hydroxy group and a hydrogen atom, respectively, while each of the remaining R-groups are hydrogen atoms.

In some embodiments, R₁₀ can be a hydrogen atom, hydroxy, a (C₁-C₆)-alkoxy group, a (C₁-C₆)-alkyl group or a halogen.

In some embodiments, R₁₀ can be a hydrogen atom, a hydroxy group, a methyl group, an ethyl group, a propyl group, a butyl group, a pentyl group, a methoxy group, an ethoxy group, a propoxy group, a butoxy group, a pentoxy group, fluorine, chlorine, bromine, or iodine.

In some embodiments, Ru can be a hydrogen atom, a hydroxy group, a (C₁-C₆)-alkoxy group, a (C₁-C₆)-alkyl group or a halogen.

In some embodiments, Ru can be a hydrogen atom, a hydroxy group, a methyl group, an ethyl group, a propyl group, a butyl group, a pentyl group, a methoxy group, an ethoxy group, a propoxy group, a butoxy group, a pentoxy group, fluorine, chlorine, bromine, or iodine.

In some embodiments, R₁₂ can be a hydrogen atom, a hydroxy group, a (C₁-C₆)-alkoxy group, a (C₁-C₆)-alkyl group or a halogen.

In some embodiments, R₁₂ can be a hydrogen atom, a hydroxy group, a methyl group, an ethyl group, a propyl group, a butyl group, a pentyl group, a methoxy group, an ethoxy group, a propoxy group, a butoxy group, a pentoxy group, fluorine, chlorine, bromine, or iodine.

In some embodiments, R₁₃ can be a hydrogen atom, a hydroxy group, a (C₁-C₆)-alkoxy group, a (C₁-C₆)-alkyl group or a halogen.

In some embodiments, R₁₃ can be a hydrogen atom, a hydroxy group, a methyl group, an ethyl group, a propyl group, a butyl group, a pentyl group, a methoxy group, an ethoxy group, a propoxy group, a butoxy group, a pentoxy group, fluorine, chlorine, bromine, or iodine.

In some embodiments, R₁₄ can be a hydrogen atom or a (C₁-C₆)-alkyl group.

In some embodiments, R₁₄ can be a hydrogen atom, a methyl group, an ethyl group, a propyl group, a butyl group or a pentyl group.

In some embodiments, R₁₀, R₁₁, R₁₂, and R₁₃ can not each simultaneously be a hydroxy group.

In some embodiments, R₁₅, R₁₆ and R₁₇ can independently or simultaneously be a hydrogen atom, a hydroxy group, a (C₁-C₆)-alkoxy group, a halogen or a (C₁-C₆)-alkyl group.

In some embodiments, R₁₅, R₁₆ and R₁₇ can independently or simultaneously be a hydrogen atom, a hydroxy group, a methoxy group, an ethoxy group, a propoxy group, a butoxy group, a pentoxy group, fluorine, chlorine, bromine, iodine, a methyl group, an ethyl group, a propyl group, a butyl group, or a pentyl group.

In some embodiments, in compound (I):
R₁ can be a hydrogen atom, or a methyl group;
R₂ can be a hydrogen atom, or a methyl group;
R₃ can be a hydrogen atom, or a methyl group;
R₄ can be a hydrogen atom, or a hydroxy group; and R₅ can be a hydrogen atom; or R₄ and R₅, taken together, can be a carbonyl group;
R₆ can be a hydrogen atom, or a methoxy group;
R₇ can be a hydrogen atom, or a methoxy group;
R₈ can be a hydrogen atom, a hydroxy group, or a methoxy group;
R₇ and R₈, taken together, can form a methylenedioxy group; and
R₉ can be a hydrogen atom, or a methyl group.

In some embodiments, in compound (II):
R₁₀ can be a hydroxy group or a methoxy group;
R₁₁ can be a hydroxy group or a methoxy group;
R₁₂ can be a hydrogen atom, a hydroxy group, or a methoxy group;
R₁₃ can be a hydroxy group or a methoxy group; and
R₁₄ can be a hydrogen atom or a methyl group.

In some embodiments, in compound (III):
R₁₅ can be a hydrogen atom or a methyl group;
R₁₆ can be a hydroxy group or a methoxy group; and
R₁₇ can be a hydroxy group, or a methoxy group;

In some embodiments, the alkaloid substrate can be 2-phenylethan-1-amine.

In some embodiments, the alkaloid substrate can be phentermine.

In some embodiments, the alkaloid substrate can be amphetamine.

In some embodiments, the alkaloid substrate can be cathinone.

In some embodiments, the alkaloid substrate can be N-methyl-cathinone.

In some embodiments, the alkaloid substrate can be nor(pseudo)ephedrine.

In some embodiments, the alkaloid substrate can be (pseudo)ephedrine.

In some embodiments, the alkaloid substrate can be methyl-(pseudo)ephedrine.

In some embodiments, the alkaloid substrate can be tyramine.

In some embodiments, the alkaloid substrate can be mescaline.

In some embodiments, the alkaloid substrate can be methylenedioxyamphetamine.

In some embodiments, the alkaloid substrate can be synephrine.

In some embodiments, the alkaloid substrate can be THQ1.

In some embodiments, the alkaloid substrate can be THQ2.

In some embodiments, the alkaloid substrate can be reticuline.

In some embodiments, the alkaloid substrate can be coclaurine.

In some embodiments, the alkaloid substrate can be papaverine.

In some embodiments, the alkaloid substrate can be stylopine.

In some embodiments, the alkaloid substrate can be tryptamine.

In some embodiments, the alkaloid substrate can be harmaline.

In some embodiments, the alkaloid substrate can be propanolol.

In some embodiments, the alkaloid substrate can comprise a primary amine and the enzyme-catalyzed N-alkylation forms an N-alkylated alkaloid product comprising a secondary amine.

In some embodiments, the alkaloid substrate can comprise a secondary amine and the enzyme-catalyzed N-alkylation forms an N-alkylated alkaloid product comprising a tertiary amine.

In some embodiments, the alkaloid substrate can comprise a tertiary amine and the enzyme-catalyzed N-alkylation forms an N-alkylated alkaloid product comprising a quaternary amine.

In another aspect, the present disclosure provides, in at least one embodiment, a method of making two alkaloids comprising:
(a) providing a first and second alkaloid substrate each independently selected from the group of substrates consisting of
   (i) a first alkaloid compound (I):

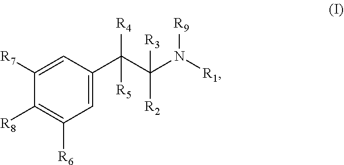

wherein:
   R₁ is a hydrogen atom, or a methyl group;
   R₂ is a hydrogen atom, or a methyl group;
   R₃ is a hydrogen atom, or a methyl group;
   R₄ is a hydrogen atom, or a hydroxy group; and R₅ is a hydrogen atom; or R₄ and R₅, taken together, are a carbonyl group;
   R₆ is a hydrogen atom, or a methoxy group;
   R₇ is a hydrogen atom, or a methoxy group;
   R₈ is a hydrogen atom, a hydroxy group, or a methoxy group;
   R₇ and R₈, taken together, form a methylenedioxy group; and
   R₉ is a hydrogen atom or a methyl group;
(ii) a second alkaloid compound (II):

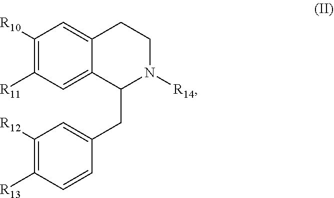

wherein in compound (II):
   R₁₀ is a hydroxy group or a methoxy group;
   R₁₁ is a hydroxy group or a methoxy group;
   R₁₂ is a hydrogen atom, a hydroxy group, or a methoxy group;
   R₁₃ is a hydroxy group or a methoxy group; and
   R₁₄ is a hydrogen atom or a methyl group;
(iii) a third alkaloid compound (III):

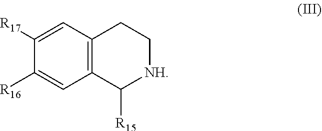

wherein in compound (III):
   R₁₅, R₁₆ and R₁₇ are independently or simultaneously a hydrogen atom, a hydroxy group, an alkoxy group, a halogen or an alkyl group; and
(iv) a fourth compound selected from stylopine; tryptamine; harmaline; and propanolol; and
(b) contacting the first and second alkaloid substrates with sufficient quantities of an alkyl donor compound and catalytic quantities of an enzyme encoded by a nucleic acid sequence selected from the group consisting of:
   (i) SEQ. ID NO: 1 or SEQ. ID NO: 6;
   (ii) a nucleic acid sequence that is substantially identical to SEQ. ID NO: 1 or SEQ. ID NO: 6;
   (iii) a nucleic acid sequence that is substantially identical to SEQ. ID NO: 1 or SEQ. ID NO: 6 but for the degeneration of the genetic code;
   (iv) a nucleic acid sequence that is complementary to SEQ. ID NO: 1 or SEQ. ID NO: 6;
   (v) a nucleic acid sequence encoding a polypeptide having the amino acid sequence set forth in SEQ. ID NO: 2 or SEQ. ID NO: 7;
   (vi) a nucleic acid sequence that encodes a functional variant of a polypeptide having the amino acid sequence set forth in SEQ. ID NO: 2 or SEQ. ID NO: 7; and
   (vii) a nucleic acid sequence that hybridizes under stringent conditions to any one of the nucleic acid sequences set forth in (i), (ii), (iii), (iv), (v) or (vi),

under reaction conditions permitting an enzyme-catalyzed N-alkylation of the two alkaloid substrates to form two N-alkylated alkaloid products.

In some embodiments, the reaction conditions can be in vitro reaction conditions.

In some embodiments, the reaction conditions can be in vivo reaction conditions.

In another aspect, the present disclosure provides, in at least one embodiment, a method for preparing an N-alkylated product alkaloid compound comprising:
(A) providing a chimeric nucleic acid sequence comprising as operably linked components:
   (a) a nucleic acid sequence encoding an N-alkyltransferase polypeptide comprising a polypeptide sequence encoded by a nucleic acid sequence selected from the group consisting of:
      (i) SEQ. ID NO: 1 or SEQ. ID NO: 6;
      (ii) a nucleic acid sequence that is substantially identical to SEQ. ID NO: 1 or SEQ. ID NO: 6;
      (iii) a nucleic acid sequence that is substantially identical to SEQ. ID NO: 1 or SEQ. ID NO: 6 but for the degeneration of the genetic code;
      (iv) a nucleic acid sequence that is complementary to SEQ. ID NO: 1 or SEQ. ID NO: 6;
      (v) a nucleic acid sequence encoding a polypeptide having the amino acid sequence set forth in SEQ. ID NO: 2 or SEQ. ID NO: 7;
      (vi) a nucleic acid sequence that encodes a functional variant of a polypeptide having the amino acid sequence set forth in SEQ. ID NO: 2 or SEQ. ID NO: 7; and
      (vii) a nucleic acid sequence that hybridizes under stringent conditions to any one of the nucleic acid sequences set forth in (i), (ii), (iii), (iv), (v) or (vi); and
   (b) one or more nucleic acid sequences capable of controlling expression in a host cell; and
(B) introducing the chimeric nucleic acid sequence into a host cell capable of producing a substrate alkaloid compound having chemical formula (I), (II), (III), stylopine; tryptamine; harmaline; and propanolol, and growing the host cell to produce the N-alkyltransferase to N-methylate the substrate alkaloid compound and produce a N-alkylated product alkaloid compound.

In some embodiments, the method can further include a step (c) comprising recovering the N-alkylated product alkaloid compound.

In another aspect, the present disclosure provides, in at least one embodiment, a substantially pure nucleic acid comprising one or more nucleic acid sequences selected from the group consisting of:
(i) SEQ. ID NO: 1 or SEQ. ID NO: 6;
(ii) a nucleic acid sequence that is substantially identical to SEQ. ID NO: 1 or SEQ. ID NO: 6;
(iii) a nucleic acid sequence that is substantially identical to SEQ. ID NO: 1 or SEQ. ID NO: 6 but for the degeneration of the genetic code;
(iv) a nucleic acid sequence that is complementary to SEQ. ID NO: 1 or SEQ. ID NO: 6;
(v) a nucleic acid sequence encoding a polypeptide having the amino acid sequence set forth in SEQ. ID NO: 2 or SEQ. ID NO: 7;
(vi) a nucleic acid sequence that encodes a functional variant of a polypeptide having the amino acid sequence set forth in SEQ. ID NO: 2 or SEQ. ID NO: 7; and
(vii) a nucleic acid sequence that hybridizes under stringent conditions to any one of the nucleic acid sequences set forth in (i), (ii), (iii), (iv), (v) or (vi).

In another aspect, the present disclosure provides, in at least one embodiment, a substantially pure polypeptide comprising:
(i) a polypeptide comprising the amino acid sequence set forth in SEQ. ID NO: 2 or SEQ. ID NO: 7;
(ii) is a polypeptide that is substantially identical to the amino acid sequence set forth in SEQ. ID NO: 2 or SEQ. ID NO: 7; or
(iii) a functional variant of a polypeptide comprising the amino acid sequences set forth in SEQ. ID NO: 2 or SEQ. ID NO: 7.

In another aspect, the present disclosure provides, in at least one embodiment, a chimeric nucleic acid sequence comprising as operably linked components:
(a) a nucleic acid sequence encoding an alkyltransferase, the nucleic acid sequence comprising one or more nucleic acid sequences selected from the group consisting of:
   (i) SEQ. ID NO: 1 or SEQ. ID NO: 6;
   (ii) a nucleic acid sequence that is substantially identical to SEQ. ID NO: 1 or SEQ. ID NO: 6;
   (iii) a nucleic acid sequence that is substantially identical to SEQ. ID NO: 1 or SEQ. ID NO: 6 but for the degeneration of the genetic code; (iv) a nucleic acid sequence that is complementary to SEQ. ID NO: 1 or SEQ. ID NO: 6;
   (v) a nucleic acid sequence encoding a polypeptide having the amino acid sequence set forth in SEQ. ID NO: 2 or SEQ. ID NO: 7;
   (vi) a nucleic acid sequence that encodes a functional variant of a polypeptide having the amino acid sequence set forth in SEQ. ID NO: 2 or SEQ. ID NO: 7; and
   (vii) a nucleic acid sequence that hybridizes under stringent conditions to any one of the nucleic acid sequences set forth in (i), (ii), (iii), (iv), (v) or (vi); and
(b) a nucleic acid sequence capable of controlling expression of the alkyltransferase in a host cell.

In another aspect, the present disclosure provides, in at least one embodiment, a recombinant expression vector comprising as operably linked components:
(a) a nucleic acid sequence capable of controlling expression in a host cell; and
(b) a nucleic acid sequence encoding an alkyltransferase, the nucleic acid sequence comprising one or more nucleic acid sequences selected from the group consisting of:
   (i) SEQ. ID NO: 1 or SEQ. ID NO: 6;
   (ii) a nucleic acid sequence that is substantially identical to SEQ. ID NO: 1 or SEQ. ID NO: 6;
   (iii) a nucleic acid sequence that is substantially identical to SEQ. ID NO: 1 or SEQ. ID NO: 6 but for the degeneration of the genetic code;
   (iv) a nucleic acid sequence that is complementary to SEQ. ID NO: 1 or SEQ. ID NO: 6;
   (v) a nucleic acid sequence encoding a polypeptide having the amino acid sequence set forth in SEQ. ID NO: 2 or SEQ. ID NO: 7;
   (vi) a nucleic acid sequence that encodes a functional variant of a polypeptide having the amino acid sequence set forth in SEQ. ID NO: 2 or SEQ. ID NO: 7; and
   (vii) a nucleic acid sequence that hybridizes under stringent conditions to any one of the nucleic acid sequences set forth in (i), (ii), (iii), (iv), (v) or (vi).

In another aspect, the present disclosure provides, in at least one embodiment, a host cell comprising a recombinant nucleic acid sequence selected from the group consisting of:
(i) SEQ. ID NO: 1 or SEQ. ID NO: 6;
(ii) a nucleic acid sequence that is substantially identical to SEQ. ID NO: 1 or SEQ. ID NO: 6;
(iii) a nucleic acid sequence that is substantially identical to SEQ. ID NO: 1 or SEQ. ID NO: 6 but for the degeneration of the genetic code;
(iv) a nucleic acid sequence that is complementary to SEQ. ID NO: 1 or SEQ. ID NO: 6;
(v) a nucleic acid sequence encoding a polypeptide having the amino acid sequence set forth in SEQ. ID NO: 2 or SEQ. ID NO: 7;
(vi) a nucleic acid sequence that encodes a functional variant of a polypeptide having the amino acid sequence set forth in SEQ. ID NO: 2 or SEQ. ID NO: 7; and
(vii) a nucleic acid sequence that hybridizes under stringent conditions to any one of the nucleic acid sequences set forth in (i), (ii), (iii), (iv), (v) or (vi).

In another aspect, the present disclosure provides, in at least one embodiment, a method of making an N-alkyltransferase, the method comprising:
(a) providing a chimeric nucleic acid sequence comprising as operably linked components:
   (I) a nucleic acid sequence encoding an N-alkyltransferase selected from the group consisting of:
      (i) SEQ. ID NO: 1 or SEQ. ID NO: 6;
      (ii) a nucleic acid sequence that is substantially identical to SEQ. ID NO: 1 or SEQ. ID NO: 6;
      (iii) a nucleic acid sequence that is substantially identical to SEQ. ID NO: 1 or SEQ. ID NO: 6 but for the degeneration of the genetic code;
      (iv) a nucleic acid sequence that is complementary to SEQ. ID NO: 1 or SEQ. ID NO: 6;
      (v) a nucleic acid sequence encoding a polypeptide having the amino acid sequence set forth in SEQ. ID NO: 2 or SEQ. ID NO: 7;
      (vi) a nucleic acid sequence that encodes a functional variant of a polypeptide having the amino acid sequence set forth in SEQ. ID NO: 2 or SEQ. ID NO: 7; and
      (vii) a nucleic acid sequence that hybridizes under stringent conditions to any one of the nucleic acid sequences set forth in (i), (ii), (iii), (iv), (v) or (vi); and
   (II) one or more nucleic acid sequences capable of controlling expression in a host cell;
(b) introducing the chimeric nucleic acid sequence into a host cell and growing the host cell to produce the alkyltransferase; and
(c) recovering the alkyltransferase from the host cell.

In another aspect the present disclosure provides, in at least one embodiment, a use of an N-alkyltransferase as a catalytic agent in a reaction to make an N-alkylated alkaloid compound from a substrate alkaloid compound, the substrate alkaloid selected from the group of substrates consisting of
(i) a first alkaloid compound (I):

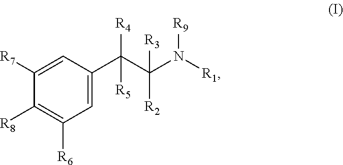

wherein
   R₁ is a hydrogen atom or an alkyl group;
   R₂ and R₃ are independently or simultaneously a hydrogen atom, a halogen or an alkyl group;
   R₄ is a hydrogen atom, a hydroxy group, a halogen, or an alkyl group;
   R₅ is a hydrogen atom, a halogen, or an alkyl group;
      or R₄ and R₅, taken together, form a carbonyl group;
   R₆ is a hydrogen atom, a halogen, an alkyl group or an alkoxy group;
   R₇ is a hydrogen atom, a halogen, an alkyl group or an alkoxy group;
   R₈ is a hydrogen atom, a hydroxy group, a halogen, an alkyl group or an alkoxy group;
   R₇ and R₈, taken together, form a methylenedioxy group; and
   R₉ is a hydrogen atom or an alkyl group;
(ii) a second alkaloid compound (II):

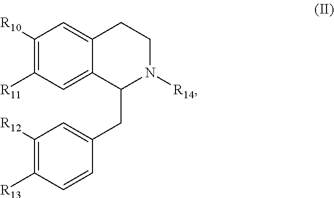

wherein
   R₁₀ is a hydrogen atom, a hydroxy group, a halogen, an alkyl group or an alkoxy group;
   R₁₁ is a hydrogen atom, a hydroxy group, a halogen, an alkyl group or an alkoxy group;
   R₁₂ is a hydrogen atom, a hydroxy group, a halogen, an alkyl group or an alkoxy group;
   R₁₃ is a hydrogen atom, a hydroxy group, a halogen, an alkyl group or an alkoxy group; and
   wherein R₁₀-R₁₃ are not each simultaneously hydroxy; and
   R₁₄ is a hydrogen atom or an alkyl group;
(iii) a third alkaloid compound (III):

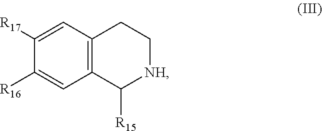

wherein
   R₁₅, R₁₆ and R₁₇ are independently or simultaneously a hydrogen atom, a hydroxy group, an alkoxy group, a halogen or an alkyl group; and
(iv) a fourth alkaloid compound selected from stylopine;
tryptamine; harmaline; and propranolol; and wherein the N-alkyltransferase:
(i) is a polypeptide comprising the amino acid sequence set forth in SEQ. ID NO: 2 or SEQ. ID NO: 7;
(ii) is a polypeptide that is substantially identical to the amino acid sequence set forth in SEQ. ID NO: 2 or SEQ. ID NO: 7; or
(iii) is a functional variant of a polypeptide comprising the amino acid sequences set forth in SEQ. ID NO: 2 or SEQ. ID NO: 7.

In another aspect, the present disclosure provides, in at least one embodiment, a pharmaceutical composition comprising an N-alkylated alkaloid compound prepared in accordance with any one of the methods of the present disclosure.

In another aspect, the present disclosure provides, in at least one embodiment, a use of an N-alkylated alkaloid compound prepared in accordance with any one of the methods of the present disclosure to prepare a pharmaceutical composition comprising the N-alkylated alkaloid compound.

In another aspect, the present disclosure provides, in at least one embodiment, a method for treating a patient with an N-alkylated alkaloid compound prepared according to the methods of the present disclosure, the method comprising administering to the patient a pharmaceutical composition comprising the N-alkylated alkaloid compound, wherein the pharmaceutical composition is administered in an amount sufficient to ameliorate a medical condition in the patient.

Other features and advantages will become apparent from the following detailed description. It should be understood, however, that the detailed description, while indicating preferred implementations of the disclosure, are given by way of illustration only, since various changes and modifications within the spirit and scope of the disclosure will become apparent to those of skill in the art from the detailed description.

### BRIEF DESCRIPTION OF THE DRAWINGS

The disclosure is in the hereinafter provided paragraphs described, by way of example, in relation to the attached figures. The figures provided herein are provided for a better understanding of the example embodiments and to show more clearly how the various embodiments may be carried into effect. The figures are not intended to limit the present disclosure.

FIG. 1 depicts the chemical structures of certain alkaloids as follows: taurine (1A), benzylamine (1B), 1-phenylethan-1-amine (1C), 2-phenylethan-1-amine (1D), phentermine (1E), amphetamine (1F), cathinone (1G), N-methyl-cathinone (1H), nor(pseudo)ephedrine (1I), pseudoephedrine (1J), methyl-(pseudo)ephedrine (1K), tyramine (1L), and mescaline (1M), methylenedioxyamphetamine (1N), and octopamine (1O).

FIG. 2 depicts the chemical structures of certain alkaloids as follows: synephrine (2A), dopamine (2B), THQ1 (2C), THQ2 (2D), norlaudanosoline (2E), reticuline (2F), coclaurine (2G), papaverine (2H), stylopine (2I), noscapine (2J), tryptamine (2K), harmaline (2L), harmine (2M), mitragynine (2N), propranolol (2O).

FIG. 3 depicts the chemical structures of certain as follows: histamine (3A), nicotinamide (3B), anthranilic acid (3C), p-dimethylaminobenzaldehyde (3D), tropinone (3E), theobromine (3F), xanthosine (3G), tyrosine (3H), 3,4-dihydroxyphenylalanine (30, phenylalanine (3J), tryptophan (3K), and adenine (3L).

FIG. 4 depicts two ion chromatograms obtained following the performance of an EsNMT catalyzed reaction to convert the N-alkylated substrate phentermine. The top panel (4A) shows an ion chromatogram obtained in a reaction mixture using native EsNMT. The bottom panel (4B) shows an ion chromatogram obtained in a reaction mixture using denatured NMT.

FIG. 5 depicts two ion chromatograms obtained following the performance of an EsNMT catalyzed reaction to convert the N-alkylated substrate amphetamine. The top panel (5A) shows an ion chromatogram obtained in a reaction mixture using native EsNMT. The bottom panel (5B) shows an ion chromatogram obtained in a reaction mixture using denatured NMT.

FIG. 6 depicts two ion chromatograms obtained following the performance of an EsNMT catalyzed reaction to convert the N-alkylated substrate cathinone. The top panel (6A) shows an ion chromatogram obtained in a reaction mixture using native EsNMT. The bottom panel (6B) shows an ion chromatogram obtained in a reaction mixture using denatured NMT.

FIG. 7 depicts two ion chromatograms obtained following the performance of an EsNMT catalyzed reaction to convert the N-alkylated substrate N-methyl-cathinone. The top panel (7A) shows an ion chromatogram obtained in a reaction mixture using native EsNMT. The bottom panel (7B) shows an ion chromatogram obtained in a reaction mixture using denatured NMT.

FIG. 8 depicts two ion chromatograms obtained following the performance of an EsNMT catalyzed reaction to convert the N-alkylated substrate nor(pseudo)ephedrine. Panel (8A) shows an ion chromatogram obtained in a reaction mixture using norpseudoephedrine and native EsNMT. Panel (8B) shows an ion chromatogram obtained in a reaction mixture using norpseudoephedrine and denatured NMT. Panel (8C) shows an ion chromatogram obtained in a reaction mixture using norephedrine and native EsNMT. Panel (8D) shows an ion chromatogram obtained in a reaction mixture using norephedrine and denatured NMT.

FIG. 9 depicts two ion chromatograms obtained following the performance of an EsNMT catalyzed reaction to convert the N-alkylated substrate (pseudo)ephedrine. Panel (9A) shows an ion chromatogram obtained in a reaction mixture using pseudoephedrine and native EsNMT. Panel (9B) shows an ion chromatogram obtained in a reaction mixture using pseudoephedrine and denatured NMT. Panel (9C) shows an ion chromatogram obtained in a reaction mixture using ephedrine and native EsNMT. Panel (9D) shows an ion chromatogram obtained in a reaction mixture using ephedrine and denatured NMT.

FIG. 10 depicts two ion chromatograms obtained following the performance of an EsNMT catalyzed reaction to convert the N-alkylated substrate tyramine. The top panel (10A) shows an ion chromatogram obtained in a reaction mixture using native EsNMT. The bottom panel (10B) shows an ion chromatogram obtained in a reaction mixture using denatured NMT.

FIG. 11 depicts two ion chromatograms obtained following the performance of an EsNMT catalyzed reaction to convert the N-alkylated substrate mescaline. The top panel (11A) shows an ion chromatogram obtained in a reaction mixture using native EsNMT. The bottom panel (11B) shows an ion chromatogram obtained in a reaction mixture using denatured NMT.

FIG. 12 depicts two ion chromatograms obtained following the performance of an EsNMT catalyzed reaction to convert the N-alkylated substrate methylenedioxyamphetamine. The top panel (12A) shows an ion chromatogram obtained in a reaction mixture using native EsNMT. The bottom panel (12B) shows an ion chromatogram obtained in a reaction mixture using denatured NMT.

FIG. 13 depicts two ion chromatograms obtained following the performance of an EsNMT catalyzed reaction to convert the N-alkylated substrate synephrine. The top panel (13A) shows an ion chromatogram obtained in a reaction mixture using native EsNMT. The bottom panel (13B) shows an ion chromatogram obtained in a reaction mixture using denatured NMT.

FIG. 14 depicts two ion chromatograms obtained following the performance of an EsNMT catalyzed reaction to convert the N-alkylated substrate THQ2. The top panel (14A) shows an ion chromatogram obtained in a reaction mixture using native EsNMT. The bottom panel (14B) shows an ion chromatogram obtained in a reaction mixture using denatured NMT.

FIG. 15 depicts two ion chromatograms obtained following the performance of an EsNMT catalyzed reaction to convert the N-alkylated substrate reticuline. The top panel (15A) shows an ion chromatogram obtained in a reaction mixture using native EsNMT. The bottom panel (15B) shows an ion chromatogram obtained in a reaction mixture using denatured NMT.

FIG. 16 depicts two ion chromatograms obtained following the performance of an EsNMT catalyzed reaction to convert the N-alkylated substrate coclaurine. The top panel (16A) shows an ion chromatogram obtained in a reaction mixture using native EsNMT. The bottom panel (16B) shows an ion chromatogram obtained in a reaction mixture using denatured NMT.

FIG. 17 depicts a pair of chromatograms obtained following the performance of an EsNMT catalyzed reaction to convert the N-alkylated substrate THQ1. The upper chromatogram (u) shows an ion chromatogram obtained in a reaction mixture using native THQ1. The lower chromatogram panel (1) shows an ion chromatogram obtained in a reaction mixture using denatured NMT. The highlighted area identifies the obtained reaction product.

FIG. 18 depicts two ion chromatograms obtained following the performance of an EsNMT catalyzed reaction to convert the N-alkylated substrate papaverine. The top panel (18A) shows an ion chromatogram obtained in a reaction mixture using native EsNMT. The bottom panel (18B) shows an ion chromatogram obtained in a reaction mixture using denatured NMT.

FIG. 19 depicts two ion chromatograms obtained following the performance of an EsNMT catalyzed reaction to convert the N-alkylated substrate tryptamine. The top panel (19A) shows an ion chromatogram obtained in a reaction mixture using native EsNMT. The bottom panel (19B) shows an ion chromatogram obtained in a reaction mixture using denatured NMT.

FIG. 20 depicts two ion chromatograms obtained following the performance of an EsNMT catalyzed reaction to convert the N-alkylated substrate harmaline. The top panel (20A) shows an ion chromatogram obtained in a reaction mixture using native EsNMT. The bottom panel shows (20B) an ion chromatogram obtained in a reaction mixture using denatured NMT.

FIG. 21 depicts two ion chromatograms obtained following the performance of an EsNMT catalyzed reaction to convert the N-alkylated substrate propanolol. The top panel (21A) shows an ion chromatogram obtained in a reaction mixture using native EsNMT. The bottom panel (21B) shows an ion chromatogram obtained in a reaction mixture using denatured NMT.

FIG. 22 depicts a pair of chromatograms obtained following the performance of an EsNMT catalyzed reaction to convert the N-alkylated substrate stylopine. The upper chromatogram (u) shows an ion chromatogram obtained in a reaction mixture using native stylopine. The lower chromatogram panel (1) shows an ion chromatogram obtained in a reaction mixture using denatured NMT. The highlighted area identifies the obtained reaction product.

FIG. 23 depicts a pair of chromatograms obtained following the performance of an EsNMT catalyzed reaction to convert the N-alkylated substrate methylephedrine. The upper chromatogram (u) shows an ion chromatogram obtained in a reaction mixture using native methylephedrine. The lower chromatogram panel (1) shows an ion chromatogram obtained in a reaction mixture using denatured NMT. The highlighted area identifies the obtained reaction product.

The figures together with the following detailed description make apparent to those skilled in the art how the disclosure may be implemented in practice.

### DETAILED DESCRIPTION OF THE DISCLOSURE

Various compositions, systems or processes will be described below to provide an example of an embodiment of each claimed subject matter. No embodiment described below limits any claimed subject matter and any claimed subject matter may cover processes, compositions or systems that differ from those described below. The claimed subject matter is not limited to compositions, processes or systems having all of the features of any one composition, system or process described below or to features common to multiple or all of the compositions, systems or processes described below. It is possible that a composition, system or process described below is not an embodiment of any claimed subject matter. Any subject matter disclosed in a composition, system or process described below that is not claimed in this document may be the subject matter of another protective instrument, for example, a continuing patent application, and the applicant(s), inventor(s) or owner(s) do not intend to abandon, disclaim or dedicate to the public any such subject matter by its disclosure in this document.

As used herein and in the claims, the singular forms, such “a”, “an” and “the” include the plural reference and vice versa unless the context clearly indicates otherwise. Throughout this specification, unless otherwise indicated, “comprise,” “comprises” and “comprising” are used inclusively rather than exclusively, so that a stated integer or group of integers may include one or more other non-stated integers or groups of integers.

The term “or” is inclusive unless modified, for example, by “either”.

When ranges are used herein for physical properties, such as molecular weight, or chemical properties, such as chemical formulae, all combinations and sub-combinations of ranges and specific embodiments therein are intended to be included. Other than in the operating examples, or where otherwise indicated, all numbers expressing quantities of ingredients or reaction conditions used herein should be understood as modified in all instances by the term “about.” The term “about” when referring to a number or a numerical range means that the number or numerical range referred to is an approximation within experimental variability (or within statistical experimental error), and thus the number or numerical range may vary between 1% and 15% of the stated number or numerical range, as will be readily recognized by context. Furthermore any range of values described herein is intended to specifically include the limiting values of the range, and any intermediate value or sub-range within the given range, and all such intermediate values and sub-ranges are individually and specifically disclosed (e.g. a range of 1 to 5 includes 1, 1.5, 2, 2.75, 3, 3.90, 4, and 5). Similarly, other terms of degree such as “substantially” and “approximately” as used herein mean a reasonable amount of deviation of the modified term such that the end result is not significantly changed. These terms of degree should be construed as including a deviation of the modified term if this deviation would not negate the meaning of the term it modifies.

Unless otherwise defined, scientific and technical terms used in connection with the formulations described herein shall have the meanings that are commonly understood by those of ordinary skill in the art. The terminology used herein is for the purpose of describing particular embodiments only, and is not intended to limit the scope of the present invention, which is defined solely by the claims.

All publications, patents and patent applications are herein incorporated by reference in their entirety to the same extent as if each individual publication, patent or patent application was specifically and individually indicated to be incorporated by reference in its entirety.

### Terms and Definitions

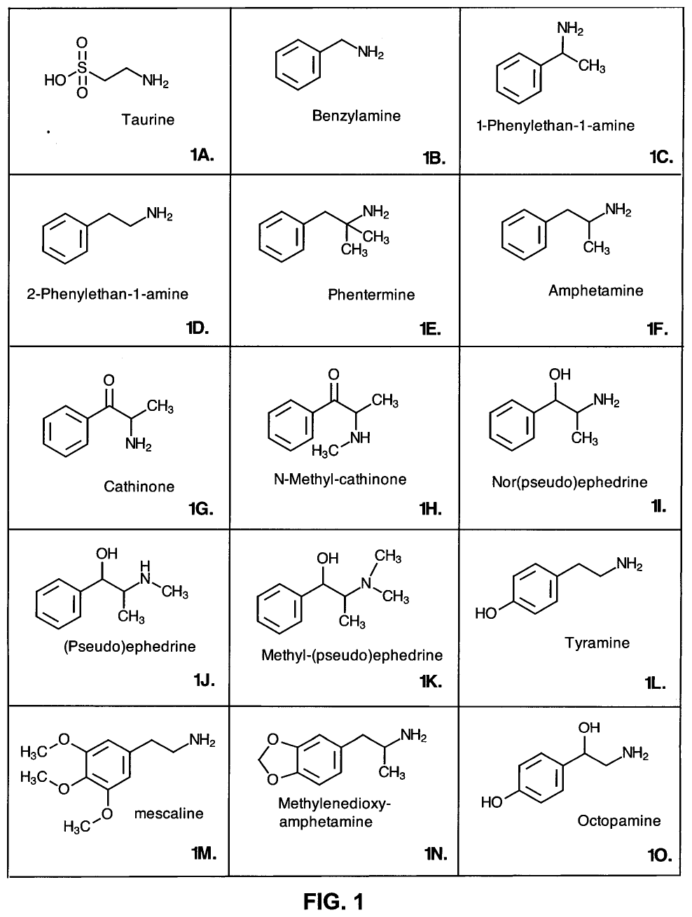

The term “taurine”, as used herein, refers to a chemical compound having the structure set forth in FIG. 1A.

The term “benzylamine”, as used herein, refers to a chemical compound having the structure set forth in FIG. 1B.

The term “1-phenylethan-1-amine”, as used herein, refers to a chemical compound having the structure set forth in FIG. 1C.

The term “2-phenylethan-1-amine”, as used herein, refers to a chemical compound having the structure set forth in FIG. 1D.

The term “phentermine”, as used herein, refers to a chemical compound having the structure set forth in FIG. 1E.

The term “amphetamine”, as used herein, refers to a chemical compound having the structure set forth in FIG. 1F.

The term “cathinone”, as used herein, refers to a chemical compound having the structure set forth in FIG. 1G.

The term “N-methyl-cathinone”, as used herein, refers to a chemical compound having the structure set forth in FIG. 1H.

The term “nor(pseudo)ephedrine”, as used herein, refers to a chemical compound having the structure set forth in FIG. 1I. The term is intended to include the stereoisomers 1S, 2R norephedrine; 1R, 2S norephedrine; 1R, 2R norpseudoephedrine; and 1S, 2S norpseudoephedrine.

The term “(pseudo)ephedrine”, as used herein, refers to a chemical compound having the structure set forth in FIG. 1J. The term is intended to include the stereoisomers 1S, 2R ephedrine; 1R, 2S ephedrine; 1R, 2R pseudoephedrine; and 1S, 2S pseudoephedrine.

The term “methyl-(pseudo)ephedrine”, as used herein, refers to a chemical compound having the structure set forth in FIG. 1K. The term is intended to include the stereoisomers 1S, 2R methylephedrine; 1R, 2S methylephedrine; 1R, 2R methylpseudoephedrine; and 1S, 2S methylpseudoephedrine.

The term “tyramine”, as used herein, refers to a chemical compound having the structure set forth in FIG. 1L.

The term “mescaline”, as used herein, refers to a chemical compound having the structure set forth in FIG. 1M.

The term “methylenedioxyamphetamine”, as used herein, refers to a chemical compound having the structure set forth in FIG. 1N.

The term “octopamine”, as used herein, refers to a chemical compound having the structure set forth in FIG. 1O.

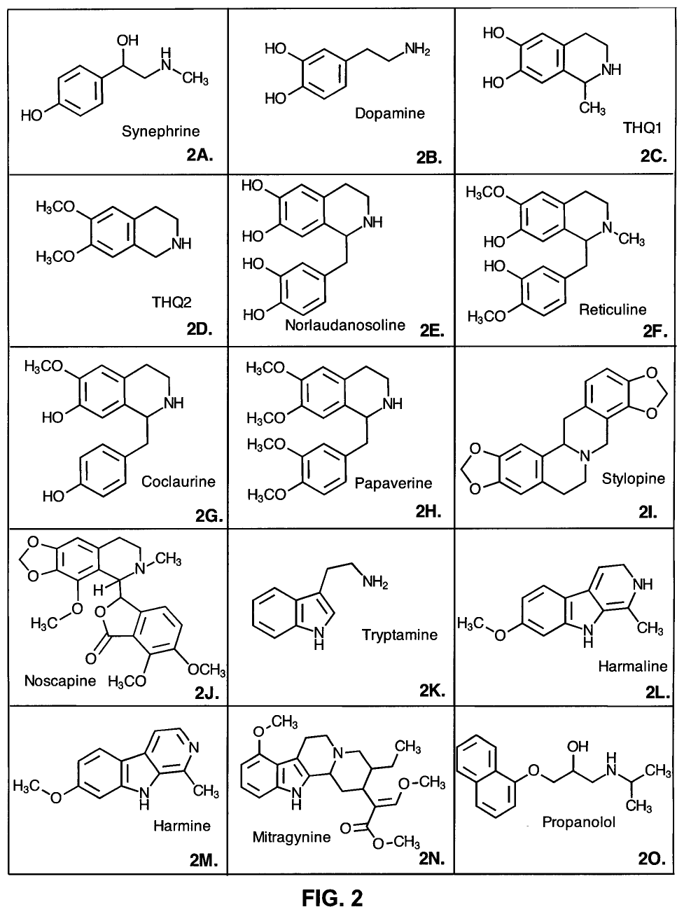

The term “synephrine”, as used herein, refers to a chemical compound having the structure set forth in FIG. 2A.

The term “dopamine”, as used herein, refers to a chemical compound having the structure set forth in FIG. 2B.

The term “THQ1”, as used herein, refers to a chemical compound, also known as 1-methyl-6,7-dihydroxy-1,2,3,4-tetrahydroisoquinoline, and having the structure set forth in FIG. 2C.

The term “THQ2”, as used herein, refers to a chemical compound, also known as 6,7-dimethoxy-1,2,3,4-tetrahydroisoquinoline, and having the structure set forth in FIG. 2D.

The term “norlaudanosoline”, as used herein, refers to a chemical compound having the structure set forth in FIG. 2E.

The term “reticuline”, as used herein, refers to a chemical compound having the structure set forth in FIG. 2F.

The term “coclaurine”, as used herein, refers to a chemical compound having the structure set forth in FIG. 2G.

The term “papaverine”, as used herein, refers to a chemical compound having the structure set forth in FIG. 2H.

The term “stylopine”, as used herein, refers to a chemical compound having the structure set forth in FIG. 2I.

The term “noscapine”, as used herein, refers to a chemical compound having the structure set forth in FIG. 2J.

The term “tryptamine”, as used herein, refers to a chemical compound having the structure set forth in FIG. 2K.

The term “harmaline”, as used herein, refers to a chemical compound having the structure set forth in FIG. 2L.

The term “harmine”, as used herein, refers to a chemical compound having the structure set forth in FIG. 2M.

The term “mitragynine”, as used herein, refers to a chemical compound having the structure set forth in FIG. 2N.

The term “propanolol”, as used herein, refers to a chemical compound having the structure set forth in FIG. 2O.

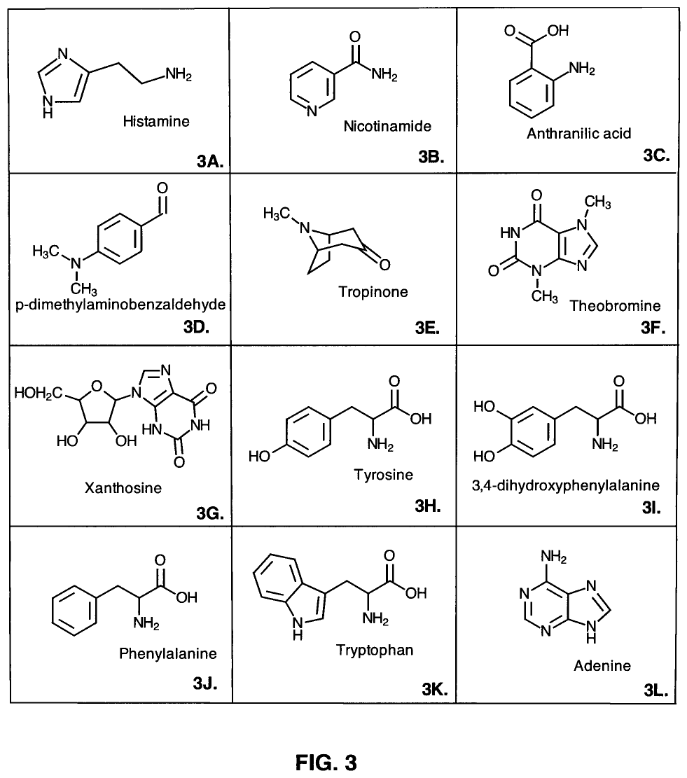

The term “histamine”, as used herein, refers to a chemical compound having the structure set forth in FIG. 3A.

The term “nicotinamide”, as used herein, refers to a chemical compound having the structure set forth in FIG. 3B.

The term “anthranilic acid”, as used herein, refers to a chemical compound having the structure set forth in FIG. 3C.

The term “p-dimethylaminobenzaldehyde”, as used herein, refers to a chemical compound having the structure set forth in FIG. 3D.

The term “tropinone”, as used herein, refers to a chemical compound having the structure set forth in FIG. 3E.

The term “theobromine”, as used herein, refers to a chemical compound having the structure set forth in FIG. 3F.

The term “xanthosine”, as used herein, refers to a chemical compound having the structure set forth in FIG. 3G.

The term “tyrosine”, as used herein, refers to a chemical compound having the structure set forth in FIG. 3H.

The term “3,4-dihydroxyphenylalanine”, as used herein, refers to a chemical compound having the structure set forth in FIG. 3I.

The term “phenylalanine”, as used herein, refers to a chemical compound having the structure set forth in FIG. 3J.

The term “tryptophan”, as used herein, refers to a chemical compound having the structure set forth in FIG. 3K.

The term “adenine”, as used herein, refers to a chemical compound having the structure set forth in FIG. 3L.

The term “alkyl” as used herein means straight and/or branched chain, saturated alkyl radicals and includes methyl, ethyl, propyl, isopropyl, n-butyl, s-butyl, isobutyl, t-butyl and the like.

The term “alkoxy” as used herein refers to alkyl groups as defined above attached to a molecule through an oxygen.

The term “nucleic acid sequence”, as used herein, refers to a sequence of nucleoside or nucleotide monomers, consisting of naturally occurring bases, sugars and intersugar (backbone) linkages. The term also includes modified or substituted sequences comprising non-naturally occurring monomers or portions thereof. The nucleic acid sequences of the present disclosure may be deoxyribonucleic nucleic acid sequences (DNA) or ribonucleic acid nucleic acid sequences (RNA) and may include naturally occurring bases including adenine, guanine, cytosine, thymidine and uracil. The nucleic acid sequences may also contain modified bases. Examples of such modified bases include aza and deaza adenine, guanine, cytosine, thymidine and uracil, and xanthine and hypoxanthine. A sequence of nucleotide or nucleoside monomers may be referred to as a nucleic acid sequence, nucleic acid sequence, a nucleotide sequence or a nucleoside sequence. The term nucleic acid sequence is deemed to be synonymous to the term nucleic acid molecule.

The term “polypeptide”, as used herein, in conjunction with a reference SEQ. ID NO, refers to any and all polypeptides comprising a sequence of amino acid residues which is (i) substantially identical to the amino acid sequence constituting the polypeptide having such reference SEQ. ID NO, or (ii) encoded by a nucleic acid sequence capable of hybridizing under at least moderately stringent conditions to any nucleic acid sequence encoding the polypeptide having such reference SEQ. ID NO, but for the use of synonymous codons. A sequence of amino acid residues may be referred to as an amino acid sequence, or polypeptide sequence.

The term “nucleic acid sequence encoding a polypeptide”, as used herein, in conjunction with a reference SEQ. ID NO, refers to any and all nucleic acid sequences encoding a polypeptide having such reference SEQ. ID NO. Nucleic acid sequences encoding a polypeptide, in conjunction with a reference SEQ. ID NO, further include any and all nucleic acid sequences which (i) encode polypeptides that are substantially identical to the polypeptide having such reference SEQ. ID NO; or (ii) hybridize to any nucleic acid sequences encoding polypeptides having such reference SEQ. ID NO under at least moderately stringent hybridization conditions or which would hybridize thereto under at least moderately stringent conditions but for the use of synonymous codons.

The term “N-alkyltransferase” refers to any and all proteins comprising a sequence of amino acid residues which is (i) substantially identical to the amino acid sequences constituting any N-alkyltransferase protein polypeptide set forth herein, including, for example, SEQ. ID NO: 2 or SEQ. ID NO: 7, or (ii) encoded by a nucleic acid sequence capable of hybridizing under at least moderately stringent conditions to any nucleic acid sequence encoding any N-alkyltransferase polypeptide set forth herein, but for the use of synonymous codons, provided however that, N-alkyltransferases, exclude any and all neopine isomerases, and further include all N-alkyltransferases set forth herein.

The terms “nucleic acid sequence encoding N-alkyltransferase”, and “nucleic acid sequence encoding an N-alkyltransferase polypeptide”, as may be used interchangeably herein, refer to any and all nucleic acid sequences encoding an N-alkyltransferase polypeptide, including, for example, SEQ. ID NO: 1 or SEQ. ID NO: 6. Nucleic acid sequences encoding an N-alkyltransferase polypeptide further include any and all nucleic acid sequences which (i) encode polypeptides that are substantially identical to N-alkyltransferase sequences set forth herein; or (ii) hybridize to any N-alkyltransferase nucleic acid sequences set forth herein under at least moderately stringent hybridization conditions, or which would hybridize thereto under at least moderately stringent conditions but for the use of synonymous codons.

By the term “substantially identical” it is meant that two amino acid sequences preferably are at least 70% identical, and more preferably are at least 85% identical and most preferably at least 95% identical, for example 96%, 97%, 98% or 99% identical. In order to determine the percentage of identity between two amino acid sequences the amino acid sequences of such two sequences are aligned, using for example the alignment method of Needleman and Wunsch (J. Mol. Biol., 1970, 48: 443), as revised by Smith and Waterman (Adv. Appl. Math., 1981, 2: 482) so that the highest order match is obtained between the two sequences and the number of identical amino acids is determined between the two sequences. Methods to calculate the percentage identity between two amino acid sequences are generally art recognized and include, for example, those described by Carillo and Lipton (SIAM J. Applied Math., 1988, 48:1073) and those described in Computational Molecular Biology, Lesk, e.d. Oxford University Press, New York, 1988, Biocomputing: Informatics and Genomics Projects. Generally, computer programs will be employed for such calculations. Computer programs that may be used in this regard include, but are not limited to, GCG (Devereux et al., Nucleic Acids Res., 1984, 12: 387) BLASTP, BLASTN and FASTA (Altschul et aL, J. Molec. Biol., 1990:215:403). A particularly preferred method for determining the percentage identity between two polypeptides involves the Clustal W algorithm (Thompson, J D, Higgines, D G and Gibson T J, 1994, Nucleic Acid Res 22(22): 4673-4680 together with the BLOSUM 62 scoring matrix (Henikoff S & Henikoff, J G, 1992, Proc. Natl. Acad. Sci. USA 89: 10915-10919 using a gap opening penalty of 10 and a gap extension penalty of 0.1, so that the highest order match obtained between two sequences wherein at least 50% of the total length of one of the two sequences is involved in the alignment.

By “at least moderately stringent hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5° C.-16.6 (Log 10 [Na+])+0.41(% (G+C)−600/l), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a >95% identity, the final wash temperature will be reduced by about 5° C. Based on these considerations those skilled in the art will be able to readily select appropriate hybridization conditions. In preferred embodiments, stringent hybridization conditions are selected. By way of example the following conditions may be employed to achieve stringent hybridization: hybridization at 5× sodium chloride/sodium citrate (SSC)/5×Denhardt's solution/1.0% SDS at Tm (based on the above equation) −5° C., followed by a wash of 0.2×SSC/0.1% SDS at 60° C. Moderately stringent hybridization conditions include a washing step in 3×SSC at 42° C. It is understood however that equivalent stringencies may be achieved using alternative buffers, salts and temperatures. Additional guidance regarding hybridization conditions may be found in: Current Protocols in Molecular Biology, John Wiley & Sons, N.Y., 1989, 6.3.1.-6.3.6 and in: Sambrook et al., Molecular Cloning, a Laboratory Manual, Cold Spring Harbor Laboratory Press, 1989, Vol. 3.

The term “functional variant”, as used herein, in reference to nucleic acid sequences or polypeptides refers to nucleic acid sequences or polypeptides capable of performing the same function as a noted reference nucleic acid sequence or polypeptide. Thus, for example, a functional variant of the polypeptide set forth in SEQ. ID NO: 2 or SEQ. ID NO: 7, refers to a polypeptide capable of performing the same function as the polypeptide set forth in SEQ. ID NO: 2 or SEQ. ID NO: 7. Functional variants include modified a polypeptide wherein, relative to a noted reference polypeptide, the modification includes a substitution, deletion or addition of one or more amino acids. In some embodiments, substitutions are those that result in a replacement of one amino acid with an amino acid having similar characteristics. Such substitutions include, without limitation (i) glutamic acid and aspartic acid; (i) alanine, serine, and threonine; (iii) isoleucine, leucine and valine, (iv) asparagine and glutamine, and (v) tryptophan, tyrosine and phenylalanine. Functional variants further include polypeptides having retained or exhibiting an enhanced alkaloid biosynthetic bioactivity.

The term “chimeric”, as used herein in the context of nucleic acid sequences and nucleic acids, refers to at least two linked nucleic acid sequences which are not naturally linked. Chimeric nucleic acid sequences or nucleic acids include linked nucleic acid sequences or nucleic acids of different natural origins. For example, a nucleic acid sequence constituting a microbial promoter linked to a nucleic acid sequence encoding a plant polypeptide is considered chimeric. Chimeric nucleic acid sequences also may comprise nucleic acid sequences of the same natural origin, provided they are not naturally linked. For example a nucleic acid sequence constituting a promoter obtained from a particular cell-type may be linked to a nucleic acid sequence encoding a polypeptide obtained from that same cell-type, but not normally linked to the nucleic acid sequence constituting the promoter. Chimeric nucleic acid sequences also include nucleic acid sequences comprising any naturally occurring nucleic acid sequences linked to any non-naturally occurring nucleic acid sequences.

The term “in vivo” as used herein to describe methods of making alkaloids refers to contacting a first alkaloid with a polypeptide capable of mediating conversion of a first alkaloid within a cell, including, for example, a microbial cell or a plant cell, to form a second alkaloid.

The term “in vitro” as used herein to describe methods of making alkaloids refers to contacting a first alkaloid with a polypeptide capable of mediating a conversion of the first alkaloid in an environment outside a cell, including, without limitation, for example, in a microwell plate, a tube, a flask, a beaker, a tank, a reactor and the like, to form a second alkaloid.

The terms “substantially pure” and “isolated”, as may be used interchangeably herein describe a compound, e.g., an alkaloid, nucleic acid sequence or a polypeptide, which has been separated from components that naturally accompany it. Typically, a compound is substantially pure when at least 60%, more preferably at least 75%, more preferably at least 90%, 95%, 96%, 97%, or 98%, and most preferably at least 99% of the total material (by volume, by wet or dry weight, or by mole percent or mole fraction) in a sample is the compound of interest. Purity can be measured by any appropriate method, e.g., in the case of polypeptides, by chromatography, gel electrophoresis or HPLC analysis.

The term “recovered” as used herein in association with an alkaloid refers to a substantially pure form of the alkaloid.

General Implementation

As hereinbefore mentioned, the present disclosure relates to alkaloids. The current disclosure further relates to certain nucleic acid sequences and polypeptides. The herein provided methods are useful in that they facilitate a novel and efficient means of making certain alkaloids, notably N-alkylated alkaloids, including, for example, N-methylated alkaloids. These methods avoid chemical synthesis of the N-alkylated alkaloids and may be conducted at commercial scale. The current disclosure further provides methodologies for the manufacture of a surprisingly wide variety of N-alkylated alkaloids, including, without limitation, a plurality of alkaloids obtainable upon alkylation of certain alkaloid compounds having the chemical structure (I), (II) or (III). The methodologies may be practiced using cells and organisms not normally capable of synthesizing the N-alkylated alkaloids. Such cells and organisms may be used as a source whence these N-alkylated alkaloids may economically be extracted. The N-alkylated alkaloids produced in accordance with the present disclosure are useful inter alia in the manufacture of pharmaceutical compositions.

Accordingly, the present disclosure provides, in at least one aspect, and in at least one embodiment, a method of making an alkaloid comprising:
(a) providing an alkaloid substrate selected from the group of substrates consisting of:
   (i) a first alkaloid compound (I):

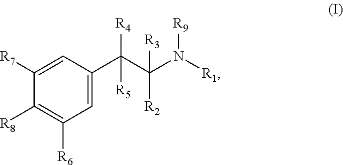

wherein
   R₁ is a hydrogen atom or an alkyl group;
   R₂ and R₃ are independently or simultaneously a hydrogen atom, a halogen or an alkyl group;
   R₄ is a hydrogen atom, a hydroxy group, a halogen, or an alkyl group;
   R₅ is a hydrogen atom, a halogen, or an alkyl group;
      or R₄ and R₅, taken together, form a carbonyl group;
   R₆ is a hydrogen atom, a halogen, an alkyl group or an alkoxy group;
   R₇ is a hydrogen atom, a halogen, an alkyl group or an alkoxy group;
   R₈ is a hydrogen atom, a hydroxy group, a halogen, an alkyl group or an alkoxy group;
   R₇ and R₈, taken together, form a methylenedioxy group; and
   R₉ is a hydrogen atom or an alkyl group;
(ii) a second alkaloid compound (II):

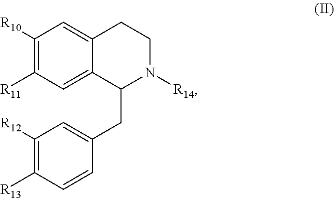

wherein
   R₁₀ is a hydrogen atom, a hydroxy group, a halogen, an alkyl group or an alkoxy group;
   R₁₁ is a hydrogen atom, a hydroxy group, a halogen, an alkyl group or an alkoxy group;
   R₁₂ is a hydrogen atom, a hydroxy group, a halogen, an alkyl group or an alkoxy group;
   R₁₃ is a hydrogen atom, a hydroxy group, a halogen, an alkyl group or an alkoxy group;
   wherein R₉-R₁₂ are not each simultaneously hydroxy;
   R₁₃ is a hydrogen atom or an alkyl group;
(iii) a third alkaloid compound (III):

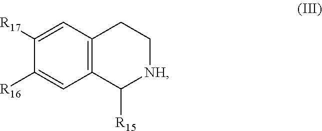

wherein
   R₁₅, R₁₆ and R₁₇ are independently or simultaneously a hydrogen atom, a hydroxy group, an alkoxy group, a halogen or an alkyl group; and
(iv) a fourth alkaloid compound selected from stylopine; tryptamine; harmaline; and propanolol; and
(b) contacting the alkaloid with sufficient quantities of an alkyl donor compound and catalytic quantities of an enzyme encoded by a nucleic acid sequence selected from the group consisting of:
   (i) SEQ. ID NO: 1 or SEQ. ID NO: 6;
   (ii) a nucleic acid sequence that is substantially identical to SEQ. ID NO: 1 or SEQ. ID NO: 6;
   (iii) a nucleic acid sequence that is substantially identical to SEQ. ID NO: 1 or SEQ. ID NO: 6 but for the degeneration of the genetic code;
   (iv) a nucleic acid sequence that is complementary to SEQ. ID NO: 1 or SEQ. ID NO: 6;
   (v) a nucleic acid sequence encoding a polypeptide having the amino acid sequence set forth in SEQ. ID NO: 2 or SEQ. ID NO: 7;
   (vi) a nucleic acid sequence that encodes a functional variant of a polypeptide having the amino acid sequence set forth in SEQ. ID NO: 2 or SEQ. ID NO: 7; and
   (vii) a nucleic acid sequence that hybridizes under stringent conditions to any one of the nucleic acid sequences set forth in (i), (ii), (iii), (iv), (v) or (vi),
   under reaction conditions permitting an enzyme-catalyzed N-alkylation of the alkaloid substrate to form a N-alkylated alkaloid product.

In some embodiments, the alkyl donor compound is a methyl donor compound and the enzyme is a methyltransferase capable of N-methylation of the alkaloid substrate to form a N-methylated alkaloid product.

In some embodiments, R₁ represents a hydrogen atom or a (C₁-C₆)-alkyl group.

In some embodiments, R₁ represents a hydrogen atom, a methyl group, an ethyl group, a propyl group, a butyl group, or a pentyl group.

In some embodiments, R₂ represents a hydrogen atom, a (C₁-C₆)-alkyl group or a halogen.

In some embodiments, R₂ represents a hydrogen atom, a methyl group, an ethyl group, a propyl group, a butyl group, a pentyl group, fluorine, chlorine, bromine, or iodine.

In some embodiments, R₃ represents a hydrogen atom, a (C₁-C₆)-alkyl group or a halogen.

In some embodiments, R₃ represents a hydrogen atom, a methyl group, an ethyl group, a propyl group, a butyl group, a pentyl group, fluorine, chlorine, bromine, or iodine.

In some embodiments, R₄ represents a hydrogen atom, a (C₁-C₆)-alkyl group or a halogen.

In some embodiments, R₄ represents a hydrogen atom, a methyl group, an ethyl group, a propyl group, a butyl group, a pentyl group, fluorine, chlorine, bromine, or iodine.

In some embodiments, R₅ represents a hydrogen atom, a (C₁-C₆)-alkyl group or a halogen.

In some embodiments, R₅ represents a hydrogen atom, a methyl group, an ethyl group, a propyl group, a butyl group, a pentyl group, fluorine, chlorine, bromine, or iodine.

In some embodiments, R₄ represents a hydrogen atom or a hydroxy group, and R₅ represents a hydrogen atom.

In some embodiments, R₄ and R₅, taken together, represents a carbonyl group.

In some embodiments, R₆ represents a hydrogen atom, a (C₁-C₆)-alkoxy group, a (C₁-C₆)-alkyl group or a halogen.

In some embodiments, R₆ represents a hydrogen atom, a methyl group, an ethyl group, a propyl group, a butyl group, a pentyl group, a methoxy group, an ethoxy group, a propoxy group, a butoxy group, a pentoxy group, fluorine, chlorine, bromine, or iodine.

In some embodiments, R₇ represents a hydrogen atom, a (C₁-C₆)-alkoxy group, a (C₁-C₆)-alkyl group or a halogen.

In some embodiments, R₇ represents a hydrogen atom, a methyl group, an ethyl group, a propyl group, a butyl group, a pentyl group, a methoxy group, an ethoxy group, a propoxy group, a butoxy group, a pentoxy group, fluorine, chlorine, bromine, or iodine.

In some embodiments, R₈ represents a hydrogen atom, a hydroxy group, a (C₁-C₆)-alkoxy group, a (C₁-C₆)-alkyl group or a halogen.

In some embodiments, R₈ represents a hydrogen atom, a hydroxy group, a methyl group, an ethyl group, a propyl group, a butyl group, a pentyl group, a methoxy group, an ethoxy group, a propoxy group, a butoxy group, a pentoxy group, fluorine, chlorine, bromine, or iodine.

In some embodiments, R₇ and R₈, taken together, form a methylenedioxy group.

In some embodiments, R₁, R₄, R₈ and R₉ can not simultaneously represent a hydrogen atom, a hydroxy group, a hydroxy group and a hydrogen atom, respectively, i.e., in alkaloid compound (I), R₁ is excluded from being a hydrogen atom, when R₄ is simultaneously a hydroxy group; Ra is simultaneously a hydroxy group; and R₉ is simultaneously a hydrogen atom. In one embodiment, the first alkaloid compound (I) is not octopamine.

In some embodiments, R₁, R₄, R₈ and R₉ can not simultaneously a hydrogen atom, a hydroxy group, a hydroxy group and a hydrogen atom, respectively, while each of the remaining R-groups are hydrogen atoms, i.e., in alkaloid compound (I) R₁ is excluded from being a hydrogen atom, when R₄ is simultaneously a hydroxy group; Ra is simultaneously a hydroxy group; and R₉ is simultaneously a hydrogen atom, while each of the remaining R-groups are hydrogen atoms. In one embodiment, the first alkaloid compound (I) is not octopamine.

In some embodiments, R₁₀ represents a hydrogen atom, a hydroxy group, a (C₁-C₆)-alkoxy group, a (C₁-C₆)-alkyl group or a halogen.

In some embodiments, R₁₀ represents a hydrogen atom, a hydroxy group, a methyl group, an ethyl group, a propyl group, a butyl group, a pentyl group, a methoxy group, an ethoxy group, a propoxy group, a butoxy group, a pentoxy group, fluorine, chlorine, bromine, or iodine.

In some embodiments, R₁₁ represents a hydrogen atom, a hydroxy group, a (C₁-C₆)-alkoxy group, a (C₁-C₆)-alkyl group or a halogen.

In some embodiments, Ru represents a hydrogen atom, a hydroxy group, a methyl group, an ethyl group, a propyl group, a butyl group, a pentyl group, a methoxy group, an ethoxy group, a propoxy group, a butoxy group, a pentoxy group, fluorine, chlorine, bromine, or iodine.

In some embodiments, R₁₂ represents a hydrogen atom, a hydroxy group, a (C₁-C₆)-alkoxy group, a (C₁-C₆)-alkyl group or a halogen.

In some embodiments, R₁ represents a hydrogen atom, a hydroxy group, a methyl group, an ethyl group, a propyl group, a butyl group, a pentyl group, a methoxy group, an ethoxy group, a propoxy group, a butoxy group, a pentoxy group, fluorine, chlorine, bromine, or iodine.

In some embodiments, R₁₃ represents a hydrogen atom, a hydroxy group, a (C₁-C₆)-alkoxy group, a (C₁-C₆)-alkyl group or a halogen.

In some embodiments, R₁₃ represents a hydrogen atom, a hydroxy group, a methyl group, an ethyl group, a propyl group, a butyl group, a pentyl group, a methoxy group, an ethoxy group, a propoxy group, a butoxy group, a pentoxy group, fluorine, chlorine, bromine, or iodine.

In some embodiments, R₁₄ represents a hydrogen atom or a (C₁-C₆)-alkyl group.

In some embodiments, R₁₄ represents a hydrogen atom, a methyl group, an ethyl group, a propyl group, a butyl group or a pentyl group.

In some embodiments, R₁₀, R₁₂, R₁₂, and R₁₃ represent each not simultaneously a hydroxy group.

In some embodiments, R₁₅, R₁₆ and R₁₇ represent independently or simultaneously a hydrogen atom, a hydroxy group, a (C₁-C₆)-alkoxy group, a halogen or a (C₁-C₆)-alkyl group.

In some embodiments, R₁₅, R₁₆ and R₁₇ represent independently or simultaneously a hydrogen atom, a hydroxy group, a methoxy group, an ethoxy group, a propoxy group, a butoxy group, a pentoxy group, fluorine, chlorine, bromine, iodine, a methyl group, an ethyl group, a propyl group, a butyl group, or a pentyl group.

In some embodiments, in compound (I):
R₁ represents a hydrogen atom, or a methyl group;
R₂ represents a hydrogen atom, or a methyl group;
R₃ represents a hydrogen atom, or a methyl group;
R₄ represents a hydrogen atom, or a hydroxy group; and R₅ represents a hydrogen atom; or R₄ and R₅, taken together, represent a carbonyl group;
R₆ represents a hydrogen atom, or a methoxy group;
R₇ represents a hydrogen atom, or a methoxy group;
R₈ represents a hydrogen atom, a hydroxy group, or a methoxy group;
R₇ and R₈, taken together, form a methylenedioxy group; and
R₉ represents a hydrogen atom, or a methyl group.

In some embodiments, in compound (II):
R₁₀ represents a hydroxy group or a methoxy group;
Ru represents a hydroxy group or a methoxy group;
R₁₂ represents a hydrogen atom, a hydroxy group, or a methoxy group;
R₁₃ represents a hydroxy group or a methoxy group; and
R₁₄ represents a hydrogen atom or a methyl group.

In some embodiments, in compound (III):
R₁₅ represents a hydrogen atom or a methyl group;
R₁₆ represents a hydroxy group or a methoxy group; and
R₁₇ represents a hydroxy group, or a methoxy group.

In some embodiments, in compound (I), (II) and (III), in compound (I):
R₁ represents a hydrogen atom, or a methyl group;
R₂ represents a hydrogen atom, or a methyl group;
R₃ represents a hydrogen atom, or a methyl group;
R₄ represents a hydrogen atom, or a hydroxy group; and R₅ represents a hydrogen atom; or R₄ and R₅, taken together, represent a carbonyl group;
R₆ represents a hydrogen atom, or a methoxy group;
R₇ represents a hydrogen atom, or a methoxy group;
R₈ represents a hydrogen atom, a hydroxy group, or a methoxy group;
R₇ and R₈, taken together, form a methylenedioxy group; and
R₉ represents a hydrogen atom, or a methyl group;
   in compound (II):
R₁₀ represents a hydroxy group or a methoxy group;
R₁₁ represents a hydroxy group or a methoxy group;
R₁₂ represents a hydrogen atom, a hydroxy group, or a methoxy group;
R₁₃ represents a hydroxy group or a methoxy group; and
R₁₄ represents a hydrogen atom or a methyl group;
and
   in compound (III):
R₁₅ represents a hydrogen atom or a methyl group;
R₁₆ represents a hydroxy group or a methoxy group; and
R₁₇ represents a hydroxy group, or a methoxy group.

In some embodiments, the alkaloid substrate is an alkaloid selected from the group consisting of 2-phenylethan-1-amine, phentermine, amphetamine, cathinone, N-methyl-cathinone, nor(pseudo)ephedrine, (pseudo) ephedrine, methyl(pseudo)ephedrine, tyramine, mescaline, methylenedioxyamphetamine, synephrine, THQ1, THQ2, reticuline, coclaurine, papaverine, stylopine, tryptamine, harmaline, and propanolol.

In some embodiments, the alkaloid substrate is 2-phenylethan-1-amine.

In some embodiments, the alkaloid substrate is phentermine.

In some embodiments, the alkaloid substrate is amphetamine.

In some embodiments, the alkaloid substrate is cathinone.

In some embodiments, the alkaloid substrate is N-methyl-cathinone.

In some embodiments, the alkaloid substrate is nor(pseudo)ephedrine.

In some embodiments, the alkaloid substrate is norephedrine.

In some embodiments, the alkaloid substrate is norpseudoephedrine.

In some embodiments, the alkaloid substrate is (pseudo)ephedrine.

In some embodiments, the alkaloid substrate is ephedrine.

In some embodiments, the alkaloid substrate is pseudoephedrine.

In some embodiments, the alkaloid substrate is methyl (pseudo)ephedrine.

In some embodiments, the alkaloid substrate is methylephedrine.

In some embodiments, the alkaloid substrate is methylpseudoephedrine.

In some embodiments, the alkaloid substrate is tyramine.

In some embodiments, the alkaloid substrate is mescaline.

In some embodiments, the alkaloid substrate is methylenedioxyamphetamine.

In some embodiments, the alkaloid substrate is synephrine.

In some embodiments, the alkaloid substrate is THQ1.

In some embodiments, the alkaloid substrate is THQ2.

In some embodiments, the alkaloid substrate is reticuline.

In some embodiments, the alkaloid substrate is coclaurine.

In some embodiments, the alkaloid substrate is papaverine.

In some embodiments, the alkaloid substrate is stylopine.

In some embodiments, the alkaloid substrate is tryptamine.

In some embodiments, the alkaloid substrate is harmaline.

In some embodiments, the alkaloid substrate is and propanolol.

In some embodiments, the alkyl donor compound is a methyl donor compound.

In some embodiments, the present disclosure provides a method of making two alkaloids comprising:
(a) providing a first and second alkaloid substrate each independently selected from the group of substrates consisting of
   (i) a first alkaloid compound (I):

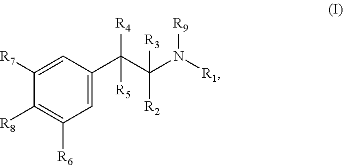

wherein:
   R₁ represents a hydrogen atom, or a methyl group;
   R₂ represents a hydrogen atom, or a methyl group;
   R₃ represents a hydrogen atom, or a methyl group;
   R₄ represents a hydrogen atom, or a hydroxy group; and R₅ represents a hydrogen atom; or R₄ and R₅, taken together, represent a carbonyl group;
   R₆ represents a hydrogen atom, or a methoxy group;
   R₇ represents a hydrogen atom, or a methoxy group; and
   R₈ represents a hydrogen atom, a hydroxy group, or a methoxy group;
   R₇ and R₈, taken together, form a methylenedioxy group; and
   R₉ is a hydrogen atom or an alkyl group;
(ii) a second alkaloid compound (II):

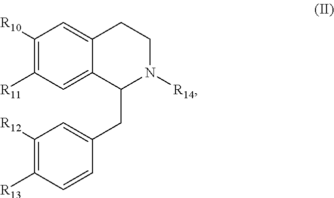

wherein in (II):
   R₁₀ represents a hydroxy group or a methoxy group;
   R₁₁ represents a hydroxy group or a methoxy group;
   R₁₂ represents a hydrogen atom, a hydroxy group, or a methoxy group;
   R₁₃ represents a hydroxy group or a methoxy group; and
   R₁₄ represents a hydrogen atom or a methyl group;
(iii) a third alkaloid compound (III):

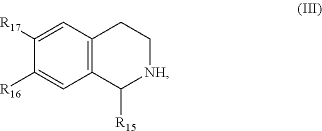

wherein
   R₁₅, R₁₆ and R₁₇ are independently or simultaneously a hydrogen atom, a hydroxy group, an alkoxy group, a halogen or an alkyl group; and
(iv) a fourth alkaloid compound selected from stylopine; tryptamine; harmaline; and propanolol; and
(b) contacting the first and second alkaloid substrates with sufficient quantities of an alkyl donor compound and catalytic quantities of an enzyme encoded by a nucleic acid sequence selected from the group consisting of:
   (i) SEQ. ID NO: 1 or SEQ. ID NO: 6;
   (ii) a nucleic acid sequence that is substantially identical to SEQ. ID NO: 1 or SEQ. ID NO: 6;
   (iii) a nucleic acid sequence that is substantially identical to SEQ. ID NO: 1 or SEQ. ID NO: 6 but for the degeneration of the genetic code;
   (iv) a nucleic acid sequence that is complementary to SEQ. ID NO: 1 or SEQ. ID NO: 6;
   (v) a nucleic acid sequence encoding a polypeptide having the amino acid sequence set forth in SEQ. ID NO: 2 or SEQ. ID NO: 7;
   (vi) a nucleic acid sequence that encodes a functional variant of a polypeptide having the amino acid sequence set forth in SEQ. ID NO: 2 or SEQ. ID NO: 7; and
   (vii) a nucleic acid sequence that hybridizes under stringent conditions to any one of the nucleic acid sequences set forth in (i), (ii), (iii), (iv), (v) or (vi),

under reaction conditions permitting an enzyme-catalyzed N-alkylation of the two alkaloid substrates to form two N-alkylated alkaloid products.

In some embodiments, the first and second alkaloid substrates are provided in a reaction mixture in such a manner that they are both simultaneously present in the same mixture. In such a reaction mixture the enzyme-catalyzed N-alkylation of the first and second alkaloid substrate can both be conducted more or less simultaneously, and the first and second alkaloid substrates can be more or less simultaneously formed.

In some embodiments, a first alkaloid substrate is provided and reacted in a reaction mixture to conduct an enzyme catalyzed N-alkylation of the first alkaloid substrate and form a first N-alkylated alkaloid product, and upon the formation of a first N-alkylated alkaloid product, a second alkaloid substrate is provided and an enzyme catalyzed N-alkylation of the second alkaloid substrate is conducted to form a second N-alkylated alkaloid product in the reaction mixture.

In some embodiments, in the performance of the reaction a substrate alkaloid compound comprising a primary amine can be converted into a product alkaloid product comprising a secondary amine in accordance with chemical reaction (i):

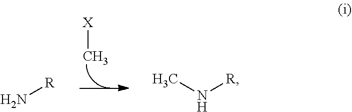

wherein X represents a carrier molecule, or a carrier molecule and an alkyl group, for example, CH₂, C₂H₄, C₃H₆ etc., wherein the carrier molecule can be any molecule which when reacted in accordance with (i) can provide an alkyl leaving group. Carrier molecules together with an alkyl group include the hereinafter mentioned alkyl-donor compounds. Thus, by way of example only, amphetamine can be converted to N-methyl-amphetamine in accordance with reaction (i)(a):

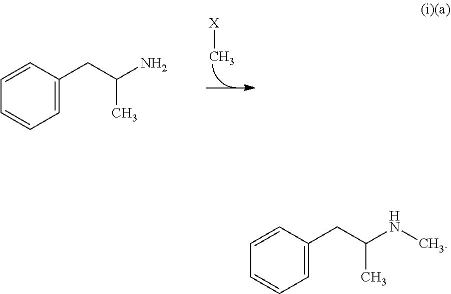

In some embodiments, in the performance of the reaction a substrate alkaloid compound comprising a secondary amine can be converted into a product alkaloid product comprising a tertiary amine in accordance with chemical reaction (ii):

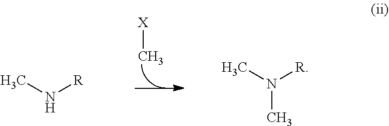

Thus, by way of example only, synephrine can be converted to N-methyl-synephrine in accordance with reaction (ii)(a):

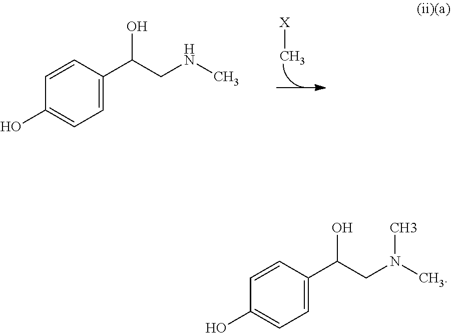

In some embodiments, in the performance of the reaction a substrate alkaloid compound a comprising tertiary amine can be converted into a product alkaloid product comprising a quaternary amine in accordance with chemical reaction (iii):

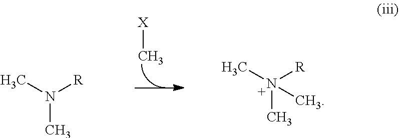

Thus, by way of example only, reticuline can be converted to N-methyl-reticuline in accordance with reaction (iii) (a)

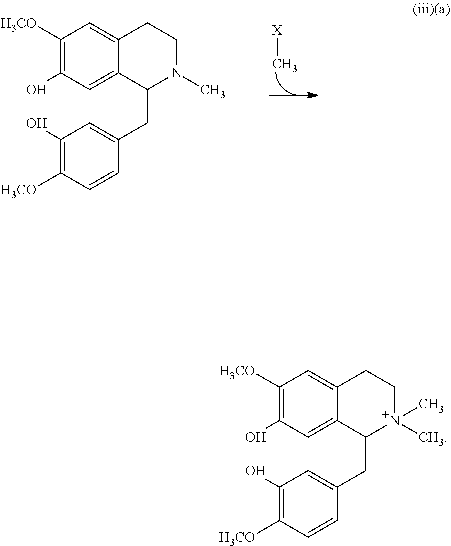

In Vitro Synthesis

In accordance with certain aspects of the present disclosure, a substrate alkaloid compound is brought in contact with sufficient quantities of an alkyl donor compound, for example a methyl donor compound, and catalytic quantities of an N-alkyltransferase, for example an N-methyltransferase, under reaction conditions permitting an enzyme catalyzed chemical conversion of the substrate alkaloid compound to form an N-alkylated alkaloid product compound under in vitro reaction conditions. Under such in vitro reaction conditions the initial reaction constituents can be provided in more or less pure form and can contacted with each other and mixed under conditions that permit the requisite chemical reactions, upon enzyme catalysis, to substantially proceed. Substantially pure forms of the substrate alkaloid compound having a chemical formula can be chemically synthesized or isolated from natural sources, including from poppy plants, including Papaver somniferum. Other plant species that may be used in accordance herewith to obtain an alkaloid substrate include, without limitation, plant species belonging to the plant families of Eupteleaceae, Lardizabalaceae, Circaeasteraceae, Menispermaceae, Berberidaceae, Ranunculaceae, and Papaveraceae (including those belonging to the subfamilies of Pteridophylloideae, Papaveroideae and Fumarioideae) and further include plants belonging to the genus Argemone, including Argemone mexicana (Mexican Prickly Poppy), plants belonging to the genus Berberis, including Berberis thunbergii (Japanese Barberry), plants belonging to the genus Chelidonium, including Chelidonium majus (Greater Celandine), plants belonging to the genus Cissampelos, including Cissampelos mucronata (Abuta), plants belonging to the genus Cocculus, including Cocculus trilobus (Korean Moonseed), plants belonging to the genus Corydalis, including Corydalis chelanthifolia (Ferny Fumewort), Corydalis cava; Corydalis ochotenis; Corydalis ophiocarpa; Corydalis platycarpa; Corydalis tuberosa; and Cordyalis bulbosa, plants belonging to the genus Eschscholzia, including Eschscholzia californica (California Poppy), plants belonging to the genus Glaucium, including Glaucium flavum (Yellowhorn Poppy), plants belonging to the genus Hydrastis, including Hydrastis canadensis (Goldenseal), plants belonging to the genus Jeffersonia, including Jeffersonia diphylla (Rheumatism Root), plants belonging to the genus Mahonia, including Mahonia aquifolium (Oregon Grape), plants belonging to the genus Menispermum, including Menispermum canadense (Canadian Moonseed), plants belonging to the genus Nandina, including Nandina domestica (Sacred Bamboo), plants belonging to the genus Nigella, including Nigella sativa (Black Cumin), plants belonging to the genus Papaver, including Papaver bracteatum (Persian Poppy), Papaver somniferum, Papaver cylindricum, Papaver decaisnei, Papaver fugax, Papaver nudicale, Papaver oreophyllum, Papaver orientale, Papaver paeonifolium, Papaver persicum, Papaver pseudo-orientale, Papaver rhoeas, Papaver rhopalothece, Papaver armeniacum, Papaver setigerum, Papaver tauricolum, and Papaver triniaefolium, plants belonging to the genus Sanguinaria, including Sanguinaria canadensis (Bloodroot), plants belonging to the genus Stylophorum, including Stylophorum diphyllum (Celandine Poppy), plants belonging to the genus Thalictrum, including Thalictrum flavum (Meadow Rue), plants belonging to the genus Tinospora, including Tinospora cordifolia (Heartleaf Moonseed), plants belonging to the genus Xanthoriza, including Xanthoriza simplicissima (Yellowroot) and plants belonging to the genus Romeria including Romeria carica.

In accordance herewith, more or less pure forms of an N-alkyltransferase enzyme may be isolated from natural sources, including microbial species, and any of the hereinbefore mentioned plant species, or they may be prepared recombinantly. Thus, provided herein is further a method for preparing an N-alkyltransferase comprising:
(a) providing a chimeric nucleic acid sequence comprising as operably linked components:
   (i) one or more nucleic acid sequences encoding an N-alkyltransferase; and
   (ii) one or more nucleic acid sequences capable of controlling expression in a host cell;
(b) introducing the chimeric nucleic acid sequence into a host cell and growing the host cell to produce an N-alkyltransferase; and
(c) recovering the N-alkyltransferase from the host cell.

In some embodiments, the N-alkyltransferase is an N-methyltransferase.

In some embodiments, N-alkyltransferase is an N-methyltransferases obtainable from Ephedra sinica.

In some embodiments, the N-alkyltransferase is an enzyme encoded by a nucleic acid sequence selected from the group consisting of:
(i) SEQ. ID NO: 1 or SEQ. ID NO: 6;
(ii) a nucleic acid sequence that is substantially identical to SEQ. ID NO: 1 or SEQ. ID NO: 6;
(iii) a nucleic acid sequence that is substantially identical to SEQ. ID NO: 1 or SEQ. ID NO: 6 but for the degeneration of the genetic code;
(iv) a nucleic acid sequence that is complementary to SEQ. ID NO: 1 or SEQ. ID NO: 6;
(v) a nucleic acid sequence encoding a polypeptide having the amino acid sequence set forth in SEQ. ID NO: 2 or SEQ. ID NO: 7;
(vi) a nucleic acid sequence that encodes a functional variant of a polypeptide having the amino acid sequence set forth in SEQ. ID NO: 2 or SEQ. ID NO: 7; and
(vii) a nucleic acid sequence that hybridizes under stringent conditions to any one of the nucleic acid sequences set forth in (i), (ii), (iii), (iv), (v) or (vi).

Growth of the host cells leads to production of the N-alkyltransferase. The polypeptides subsequently can be recovered, isolated and separated from other host cell components by a variety of different protein purification techniques including, e.g. ion-exchange chromatography, size exclusion chromatography, affinity chromatography, hydrophobic interaction chromatography, reverse phase chromatography, gel filtration, etc. Further general guidance with respect to protein purification may for example be found in: Cutler, P. Protein Purification Protocols, Humana Press, 2004, Second Ed. Thus substantially pure preparations of the N-alkyltransferase polypeptides may be obtained.

Accordingly, the present disclosure provides, in a further embodiment, a substantially pure alkyl-transferase polypeptide comprising:
(i) a polypeptide comprising the amino acid sequence set forth in SEQ. ID NO: 2 or SEQ. ID NO: 7;
(ii) is a polypeptide that is substantially identical to the amino acid sequence set forth in SEQ. ID NO: 2 or SEQ. ID NO: 7; or
(iii) a functional variant of a polypeptide comprising the amino acid sequences set forth in SEQ. ID NO: 2 or SEQ. ID NO: 7.

In accordance herewith, a substrate alkaloid compound is brought in contact with sufficient quantities of a methyl-donor compound and catalytic quantities of N-alkyltransferase under reaction conditions permitting an enzyme catalyzed chemical conversion of the substrate alkaloid compound having chemical formula to form an N-alkylated product alkaloid. A variety of alkyl-donor compounds can be used. In some embodiments, the alkyl donor compound is a methyl donor compound. In preferred embodiments, S-adenosyl methionine (SAM) can be used as a methyl-donor. In other embodiments, other methyl donors can be used including, natural or synthetic methyl-donors, including, without limitation, L-methionine; L-methionine ethyl ester (MEE); methyl ester of methionine (MME); N-derivatized methionine analogues, such as N-acetyl-L-methionine (NAM), and N,N-dimethyl-L-methionine (DMM); aziridinium-based SAM analogues. SAM analogues comprising a substituted L-methyl-group can also be used, for example, a terminal alkyl, keto or amino group; or S/Se-Met analogues. Further reference to these and other alkyl and methyl donors that can be used in accordance herewith can be found in Biochemistry (2014) 53:1521-1526; Microbiology (2015) 161 (Pt 3):674-682; Agnew. Chem. Int. Ed. (2014) 53:3965-3969; Nature Chemical Biology (2006) 2:31-32; Org. Biomol. Chem. (2013) 11:7606-7610; and Anal. Biochem. (2014) 450:11-19. The quantities of alkyl or methyl-donor that are used may vary. In some embodiments, equimolar, or approximately equimolar amounts of a methyl-donor and the substrate alkaloid compound can be provided.

In some embodiments, the agents are brought in contact with each other and mixed to form a mixture. In some embodiments, the mixture is an aqueous mixture comprising water and further optionally additional agents to facilitate enzyme catalysis, including buffering agents, salts, pH modifying agents, or other enzymes. The reaction may be performed at a range of different temperatures using catalytic quantities of the enzyme. In preferred embodiments, the reaction is performed at a temperature between about 18° C. and 60° C., or between about 37° C. and 55° C., or at around 50° C. Upon completion of the in vitro reaction the N-alkylated alkaloid product may be obtained in more or less pure form.

In Vivo Synthesis

In accordance with certain aspects of the present disclosure, a substrate alkaloid compound is brought in contact with sufficient quantities of a methyl-donor and catalytic quantities of an N-alkyltransferase under reaction conditions permitting an enzyme catalyzed chemical conversion of the substrate alkaloid compound to form an N-alkylated alkaloid product compound under in vivo reaction conditions. Under such in vivo reaction conditions living cells are modified in such a manner that they produce a product alkaloid compound. In certain embodiments, the living cells can be microorganisms, including bacterial cells and fungal cells. In other embodiments, the living cells are multicellular organisms, including plants.

In one embodiment, the living cells, for example microbial cells, can be selected to be host cells capable of producing a substrate alkaloid compound, but not a product alkaloid compound. In some embodiments, the living cells can be selected to be host cells capable of producing a substrate alkaloid compound having formula (I,); (II); (III); or stylopine; tryptamine; harmaline; and propanolol, but not a N-alkylated product alkaloid compound of any of the foregoing substrate alkaloid compounds. Such cells include, without limitation, bacteria, yeast, other fungal cells, plant cells, or animal cells. Thus, by way of example only, a host cell can be a yeast host cell capable of producing a substrate alkaloid compound having formula having formula (I,); (II); (III) or stylopine; tryptamine; harmaline; and propanolol, but not a N-alkylated product alkaloid compound of any of the foregoing. In order to modulate such host cells in such a manner that they produce an N-alkylated product alkaloid compound, an N-alkyltransferase in accordance herewith can be heterologously introduced and expressed in the host cells.

In some embodiments, the living cells naturally produce an N-alkylated product alkaloid compound obtainable upon N-alkylation of substrate alkaloid compound having formula (I,); (II); (III) or stylopine; tryptamine; harmaline; and propanolol, however the living cells are modulated in such a manner that the level of the N-alkylated product is modulated, relative to the level produced by the cell without heterologous introduction of any of the aforementioned enzymes in such living cells.

In order to produce an N-alkylated product alkaloid compound, provided herein is further a method for preparing an N-alkylated product alkaloid compound comprising:
(a) providing a chimeric nucleic acid sequence comprising as operably linked components:
   (i) a nucleic acid sequence encoding an N-alkyltransferase polypeptide; and
   (ii) one or more nucleic acid sequences capable of controlling expression in a host cell; and
(b) introducing the chimeric nucleic acid sequence into a host cell capable of producing a substrate alkaloid compound having chemical formula (I), (II), (III) or stylopine; tryptamine; harmaline; and propanolol, and growing the host cell to produce the N-alkyltransferase to N-methylate the substrate alkaloid compound and produce a N-alkylated product alkaloid compound.

In some embodiments, the method further includes a step (c) comprising recovering the N-alkylated product alkaloid compound.

In some embodiments, the nucleic acid sequences can be isolated from any of the hereinbefore mentioned plant species. In some embodiments, the N-alkyltransferase is an N-methyltransferase. In some embodiments, the N-alkyltransferase is an enzyme encoded by a nucleic acid sequence selected from the group consisting of:
(i) SEQ. ID NO: 1 or SEQ. ID NO: 6;
(ii) a nucleic acid sequence that is substantially identical to SEQ. ID NO: 1 or SEQ. ID NO: 6;
(iii) a nucleic acid sequence that is substantially identical to SEQ. ID NO: 1 or SEQ. ID NO: 6 but for the degeneration of the genetic code;
(iv) a nucleic acid sequence that is complementary to SEQ. ID NO: 1 or SEQ. ID NO: 6;
(v) a nucleic acid sequence encoding a polypeptide having the amino acid sequence set forth in SEQ. ID NO: 2 or SEQ. ID NO: 7;
(vi) a nucleic acid sequence that encodes a functional variant of a polypeptide having the amino acid sequence set forth in SEQ. ID NO: 2 or SEQ. ID NO: 7; and
(vii) a nucleic acid sequence that hybridizes under stringent conditions to any one of the nucleic acid sequences set forth in (i), (ii), (iii), (iv), (v) or (vi).

Accordingly, in another aspect, the present disclosure provides, in at least one embodiment, a substantially pure nucleic acid comprising one or more nucleic acid sequences selected from the group consisting of:
(i) SEQ. ID NO: 1 or SEQ. ID NO: 6;
(ii) a nucleic acid sequence that is substantially identical to SEQ. ID NO: 1 or SEQ. ID NO: 6;
(iii) a nucleic acid sequence that is substantially identical to SEQ. ID NO: 1 or SEQ. ID NO: 6 but for the degeneration of the genetic code;
(iv) a nucleic acid sequence that is complementary to SEQ. ID NO: 1 or SEQ. ID NO: 6;
(v) a nucleic acid sequence encoding a polypeptide having the amino acid sequence set forth in SEQ. ID NO: 2 or SEQ. ID NO: 7;
(vi) a nucleic acid sequence that encodes a functional variant of a polypeptide having the amino acid sequence set forth in SEQ. ID NO: 2 or SEQ. ID NO: 7; and
(vii) a nucleic acid sequence that hybridizes under stringent conditions to any one of the nucleic acid sequences set forth in (i), (ii), (iii), (iv), (v) or (vi).

In accordance herewith, the nucleic acid sequence encoding N-alkyltransferase can be linked to a nucleic acid sequence capable of controlling expression of N-alkyltransferase in a host cell. Accordingly, the present disclosure also provides a nucleic acid sequence encoding a N-alkyltransferase linked to a nucleic acid sequence capable of controlling expression in a host cell, and the disclosure includes, in a further embodiment, a chimeric nucleic acid sequence comprising as operably linked components:
(a) a nucleic acid sequence encoding an alkyltransferase, the nucleic acid sequence comprising one or more nucleic acid sequences selected from the group consisting of:
   (i) SEQ. ID NO: 1 or SEQ. ID NO: 6;
   (ii) a nucleic acid sequence that is substantially identical to SEQ. ID NO: 1 or SEQ. ID NO: 6;
   (iii) a nucleic acid sequence that is substantially identical to SEQ. ID NO: 1 or SEQ. ID NO: 6 but for the degeneration of the genetic code;
   (iv) a nucleic acid sequence that is complementary to SEQ. ID NO: 1 or SEQ. ID NO: 6;
   (v) a nucleic acid sequence encoding a polypeptide having the amino acid sequence set forth in SEQ. ID NO: 2 or SEQ. ID NO: 7;
   (vi) a nucleic acid sequence that encodes a functional variant of a polypeptide having the amino acid sequence set forth in SEQ. ID NO: 2 or SEQ. ID NO: 7; and
   (vii) a nucleic acid sequence that hybridizes under stringent conditions to any one of the nucleic acid sequences set forth in (i), (ii), (iii), (iv), (v) or (vi).
(b) a nucleic acid sequence capable of controlling expression of the alkyltransferase in a host cell.

Nucleic acid sequences capable of controlling expression in host cells that may be used herein include any transcriptional promoter capable of controlling expression of polypeptides in host cells. Generally, promoters obtained from bacterial cells are used when a bacterial host is selected in accordance herewith, while a fungal promoter will be used when a fungal host is selected, a plant promoter will be used when a plant cell is selected, and so on. Further nucleic acid elements capable elements of controlling expression in a host cell include transcriptional terminators, enhancers and the like, all of which may be included in the chimeric nucleic acid sequences of the present disclosure.

In accordance with the present disclosure, the chimeric nucleic acid sequences comprising a promoter capable of controlling expression in host cell linked to a nucleic acid sequence encoding an N-alkyltransferase, can be integrated into a recombinant expression vector which ensures good expression in the host cell. Accordingly, the present disclosure includes a recombinant expression vector comprising as operably linked components:
(i) a nucleic acid sequence capable of controlling expression in a host cell; and
(ii) a nucleic acid sequence encoding an N-alkyltransferase,

wherein the expression vector is suitable for expression in a host cell. The term “suitable for expression in a host cell” means that the recombinant expression vector comprises the chimeric nucleic acid sequence of the present disclosure linked to genetic elements required to achieve expression in a host cell. Genetic elements that may be included in the expression vector in this regard include a transcriptional termination region, one or more nucleic acid sequences encoding marker genes, one or more origins of replication and the like. In preferred embodiments, the expression vector further comprises genetic elements required for the integration of the vector or a portion thereof in the host cell's genome, for example if a plant host cell is used the T-DNA left and right border sequences which facilitate the integration into the plant's nuclear genome.

Pursuant to the present disclosure, the expression vector may further contain a marker gene. Marker genes that may be used in accordance with the present disclosure include all genes that allow the distinction of transformed cells from non-transformed cells, including all selectable and screenable marker genes. A marker gene may be a resistance marker such as an antibiotic resistance marker against, for example, kanamycin or ampicillin. Screenable markers that may be employed to identify transformants through visual inspection include β-glucuronidase (GUS) (U.S. Pat. Nos. 5,268,463 and 5,599,670) and green fluorescent protein (GFP) (Niedz et al., 1995, Plant Cell Rep., 14: 403).

Thus in accordance with the foregoing, the present disclosure provides, in accordance with a further embodiment, a recombinant expression vector comprising as operably linked components:
(a) a nucleic acid sequence capable of controlling expression in a host cell; and
(b) a nucleic acid sequence encoding an alkyltransferase, the nucleic acid sequence comprising one or more nucleic acid sequences selected from the group consisting of:
   (i) SEQ. ID NO: 1 or SEQ. ID NO: 6;
   (ii) a nucleic acid sequence that is substantially identical to SEQ. ID NO: 1 or SEQ. ID NO: 6;
   (iii) a nucleic acid sequence that is substantially identical to SEQ. ID NO: 1 or SEQ. ID NO: 6 but for the degeneration of the genetic code;
   (iv) a nucleic acid sequence that is complementary to SEQ. ID NO: 1 or SEQ. ID NO: 6;
   (v) a nucleic acid sequence encoding a polypeptide having the amino acid sequence set forth in SEQ. ID NO: 2 or SEQ. ID NO: 7;
   (vi) a nucleic acid sequence that encodes a functional variant of a polypeptide having the amino acid sequence set forth in SEQ. ID NO: 2 or SEQ. ID NO: 7; and
   (vii) a nucleic acid sequence that hybridizes under stringent conditions to any one of the nucleic acid sequences set forth in (i), (ii), (iii), (iv), (v) or (vi).

One host cell that particularly conveniently may be used is Escherichia coli. The preparation of the E. coli vectors may be accomplished using commonly known techniques such as restriction digestion, ligation, gelelectrophoresis, DNA sequencing, the Polymerase Chain Reaction (PCR) and other methodologies. A wide variety of cloning vectors is available to perform the necessary steps required to prepare a recombinant expression vector. Among the vectors with a replication system functional in E. coli, are vectors such as pBR322, the pUC series of vectors, the M13 mp series of vectors, pBluescript etc. Typically, these cloning vectors contain a marker allowing selection of transformed cells. Nucleic acid sequences may be introduced in these vectors, and the vectors may be introduced in E. coli by preparing competent cells, electroporation or using other well known methodologies to a person of skill in the art. E. coli may be grown in an appropriate medium, such as Luria-Broth medium and harvested. Recombinant expression vectors may readily be recovered from cells upon harvesting and lysing of the cells. Further, general guidance with respect to the preparation of recombinant vectors and growth of recombinant organisms may be found in, for example: Sambrook et al., Molecular Cloning, a Laboratory Manual, Cold Spring Harbor Laboratory Press, 2001, Third Ed.

Further included in the present disclosure are a host cell wherein the host cell comprises a heterologously introduced (i.e. recombinant) nucleic acid sequence encoding an N-alkyltransferase. Thus, in a further embodiment, the present disclosure provides, a host cell comprising a recombinant nucleic acid sequence selected from the group consisting of:
(i) SEQ. ID NO: 1 or SEQ. ID NO: 6;
(ii) a nucleic acid sequence that is substantially identical to SEQ. ID NO: 1 or SEQ. ID NO: 6;
(iii) a nucleic acid sequence that is substantially identical to SEQ. ID NO: 1 or SEQ. ID NO: 6 but for the degeneration of the genetic code;
(iv) a nucleic acid sequence that is complementary to SEQ. ID NO: 1 or SEQ. ID NO: 6;
(v) a nucleic acid sequence encoding a polypeptide having the amino acid sequence set forth in SEQ. ID NO: 2 or SEQ. ID NO: 7;
(vi) a nucleic acid sequence that encodes a functional variant of a polypeptide having the amino acid sequence set forth in SEQ. ID NO: 2 or SEQ. ID NO: 7; and
(vii) a nucleic acid sequence that hybridizes under stringent conditions to any one of the nucleic acid sequences set forth in (i), (ii), (iii), (iv), (v) or (vi).

In some embodiments, the nucleic acid sequence encoding the N-alkyltransferase is linked to one or more a nucleic acid sequences capable of controlling expression in the 5′ to 3′ direction of the N-alkyltransferase. Thus the host cell, in some embodiments can contain a chimeric nucleic acid sequence comprising a nucleic acid encoding an N-alkyltransferase, linked to one or more a nucleic acid sequences capable of controlling expression in the 5′ to 3′ direction of the N-alkyltransferase.

As hereinbefore mentioned the host cell is preferably a host cell capable of producing an alkaloid substrate having chemical formula (I,); (II); (III) or stylopine; tryptamine; harmaline; and propanolol, but not a N-alkylated product alkaloid compound of any of the foregoing substrate alkaloid compounds, but for the introduction of the chimeric nucleic acid sequences of the present disclosure.

As hereinbefore mentioned, in other embodiments, the host cells naturally produce an N-alkylated product alkaloid obtainable following alkylation of a substrate alkaloid compound having chemical formula (I,); (II); (III) or stylopine; tryptamine; harmaline; and propanolol, however the host cells are modulated in such a manner that the levels of the product alkaloid produced in the cells is modulated, relative to levels of such alkaloid produced by the cell without heterologous introduction of any of the aforementioned enzymes in such host cells. Such modulations may be achieved by a variety of modification techniques, including, but not limited to, the modulation of the enzymatic activity of an N-alkyltransferase, for example by modulating the native nucleic acid sequences encoding the N-alkyltransferase, for example by gene silencing methodologies, such as antisense methodologies; or by the use of modification techniques resulting in modulation of activity of the enzymes using for example site directed mutagenesis, targeted mutagenesis, random mutagenesis, virus-induced gene silencing, the addition of organic solvents, gene shuffling or a combination of these and other techniques known to those of skill in the art, each methodology designed to alter the activity of the enzymes of the N-alkyltransferase, in such a manner that level of product alkaloid compound in the host cells increases.

Uses of N-Alkyltransferases and N-Alkylated Product Alkaloids

As will be clear from the foregoing, in a general sense the N-alkyltransferases of the present disclosure can be used as catalytic agents to make N-alkylated alkaloid compounds. Thus, in another aspect the present disclosure further provides, a use of an N-alkyltransferase as a catalytic agent in a reaction to make an N-alkylated alkaloid compound from a substrate alkaloid compound, the substrate alkaloid selected from the group of substrates consisting of
(i) a first alkaloid compound (I):

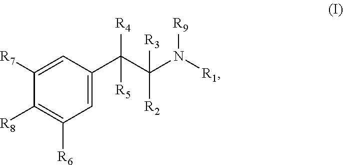

wherein
   R₁ is a hydrogen atom or an alkyl group;
   R₂ and R₃ are independently or simultaneously a hydrogen atom, a halogen or an alkyl group;
   R₄ is a hydrogen atom, a hydroxy group, a halogen, or an alkyl group;
   R₅ is a hydrogen atom, a halogen, or an alkyl group;
      or R₄ and R₅, taken together, form a carbonyl group;
   R₆ is a hydrogen atom, a halogen, an alkyl group or an alkoxy group;
   R₇ is a hydrogen atom, a halogen, an alkyl group or an alkoxy group;
   R₈ is a hydrogen atom, a hydroxy group, a halogen, an alkyl group or an alkoxy group;
   R₇ and R₈, taken together, form a methylenedioxy group; and
   R₉ is a hydrogen atom or an alkyl group;
(ii) a second alkaloid compound (II):

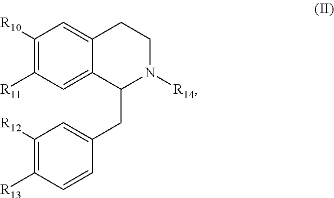

wherein
   R₁₀ is a hydrogen atom, a hydroxy group, a halogen, an alkyl group or an alkoxy group;
   R₁₁ is a hydrogen atom, a hydroxy group, a halogen, an alkyl group or an alkoxy group;
   R₁₂ is a hydrogen atom, a hydroxy group, a halogen, an alkyl group or an alkoxy group;
   R₁₃ is a hydrogen atom, a hydroxy group, a halogen, an alkyl group or an alkoxy group; and
   wherein R₁₀-R₁₃ are not each simultaneously hydroxy; and
   R₁₄ is a hydrogen atom or an alkyl group;
(iii) a third alkaloid compound (III):

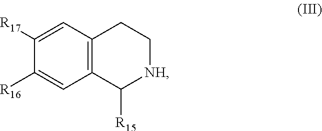

wherein
   R₁₅, R₁₆ and R₁₇ are independently or simultaneously a hydrogen atom, a hydroxy group, an alkoxy group, a halogen or an alkyl group; and
(iv) a fourth alkaloid compound selected from stylopine; tryptamine; harmaline; and propranolol; and
   wherein the N-alkyltransferase:
(i) is a polypeptide comprising the amino acid sequence set forth in SEQ. ID NO: 2 or SEQ. ID NO: 7;
(ii) is a polypeptide that is substantially identical to the amino acid sequence set forth in SEQ. ID NO: 2 or SEQ. ID NO: 7; or
(iii) is a functional variant of a polypeptide comprising the amino acid sequences set forth in SEQ. ID NO: 2 or SEQ. ID NO: 7.

The N-alkylated products obtained in accordance with the present disclosure may be formulated for use as a pharmaceutical drug, therapeutic agent or medicinal agent. Thus the present disclosure further includes a pharmaceutical composition comprising an N-alkylated product prepared in accordance with the methods of the present disclosure. Pharmaceutical drug preparations comprising an N-alkylated product in accordance with the present disclosure can comprise vehicles, excipients and auxiliary substances, such as wetting or emulsifying agents, pH buffering substances and the like. These vehicles, excipients and auxiliary substances are generally pharmaceutical agents that may be administered without undue toxicity. Pharmaceutically acceptable excipients include, but are not limited to, liquids such as water, saline, polyethyleneglycol (PEG), hyaluronic acid, glycerol and ethanol. Pharmaceutically acceptable salts can also be included therein, for example, mineral acid salts such as hydrochlorides, phosphates, sulfates, and the like; and the salts of organic acids such as acetates, propionates, benzoates, and the like. It is also preferred, although not required, that the preparation will contain a pharmaceutically acceptable excipient that serves as a stabilizer. Examples of suitable carriers that also act as stabilizers for peptides include, without limitation, pharmaceutical grades of dextrose, sucrose, lactose, sorbitol, inositol, dextran, and the like. Other suitable carriers include, again without limitation, starch, cellulose, sodium or calcium phosphates, citric acid, glycine, polyethylene glycols, and combinations thereof. The pharmaceutical composition may be formulated for oral and intravenous administration and other routes of administration as desired. Dosing may vary and may be optimized using routine experimentation.

In further embodiments, the present disclosure provides methods for treating a patient with a pharmaceutical composition comprising an N-alkylated product prepared in accordance with the present disclosure. Accordingly, the present disclosure further provides a method for treating a patient with an N-alkylated product prepared according to the methods of the present disclosure, said method comprising administering to the patient a composition comprising an N-alkylated product, wherein the N-alkylated is administered in an amount sufficient to ameliorate a medical condition in the patient.

In yet further embodiments, the present disclosure provides a use of an N-alkylated product prepared in accordance with the methods of the present disclosure to treat a patient, wherein the N-alkylated is administered in an amount sufficient to ameliorate a medical condition in the patient.

Summary of Sequences

SEQ. ID NO: 1 sets forth a nucleic acid sequence encoding an EsNMT polypeptide.

SEQ. ID NO: 2 sets forth a deduced amino acid sequence of a polypeptide sequence encoding an EsNMT polypeptide.

SEQ. ID NO: 3 sets forth a deduced amino acid sequence of a polypeptide sequence encoding a GfCNMT polypeptide.

SEQ. ID NO: 4 sets forth a deduced amino acid sequence of a polypeptide sequence encoding a GfTNMT polypeptide.

SEQ. ID NO: 5 sets forth a deduced amino acid sequence of a polypeptide sequence encoding a PsRNMT polypeptide.

SEQ. ID NO: 6 sets forth a nucleic acid sequence encoding an EsNMT polypeptide.

SEQ. ID NO: 7 sets forth a deduced amino acid sequence of a polypeptide sequence encoding an EsNMT polypeptide.

### EXAMPLES

Hereinafter are provided examples of specific implementations for performing the methods of the present disclosure, as well as implementations representing the compositions of the present disclosure. The examples are provided for illustrative purposes only, and are not intended to limit the scope of the present disclosure in any way.

### Example 1—N-Alkylation Using EsNMT, GfCNMT, GfTNMT and PsRNMT

The EsNMT coding sequence was amplified from Ephedra sinica cDNA using gene-specific primers containing restriction sites with Q5 HiFi DNA polymerase (NEB). Restriction digests and ligations (NEB) were carried out to generate a pQE plasmid (Qiagen) for heterologous expression of HIS₆-tagged recombinant EsNMT. Protein overexpression was carried out at 18° C. in Escherichia coli SG13009 (Qiagen) grown in lysogeny broth (LB) to which 0.5 mM isopropyl β-D-1-thiogalactopyranoside (IPTG) was added.

The GfCNMT, GfTNMT and PsRNMT coding sequences were amplified from Glaucium flavum (GfCNMT and GfTNMT) or Papaver somniferum (PsRNMT) cDNA using gene-specific primers containing attB recombination sites with Q5 HiFi DNA polymerase (NEB). Recombination reactions were carried out using BP and LR Clonase II (Thermo Scientific) to generate individual pDONR221 entry plasmids and, subsequently, individual pHGWA plasmids for heterologous expression of polyhistidine (HIS₆)-tagged recombinant GfCNMT, GfTNMT and PsRNMT. Protein overexpression was carried out at 18° C. using E. coli ArcticExpress (Agilent) grown in Studier's autoinduction media.

Total soluble protein was extracted from each culture and the recombinant NMTs were purified using TALON cobalt affinity resin (Clontech) followed by desalting and concentration with Amicon Ultra-30 centrifugal filters (EMD Millipore). Protein concentration was determined using the Bradford method, and purity was assessed by SDS-PAGE followed by Coomassie staining.

Forty-four nitrogenous molecules were screened as potential substrates for EsNMT using a standardized assay (25 μg protein, 1 mM substrate, 1 mM SAM, 60 mM HEPES pH 8, 16 hours at 37° C.). In addition, a representative subset was screened as potential substrates for GfCNMT, GfTNMT and PsRNMT under identical conditions. For each substrate and enzyme combination, negative control assays were carried out using protein heated to 100° C. (boiled) for 20 minutes. Assays were quenched with 9 volumes of methanol, centrifuged to pellet insoluble debris and the supernatants were stored at −20° C. prior to analysis.

Assay supernatants were analyzed by liquid chromatography-mass spectrometry (LC-MS). LC-MS analysis was performed using a 1200 HPLC coupled with a 6410 triple-quadrupole MS (Agilent). Samples (2 μl) were injected onto a Luna 5 μM Phenyl-Hexyl 100 Å HPLC column and analytes were eluted in a gradient of solvent A (0.1% Formic acid, 5% v/v methanol in water) and solvent B (0.1% Formic acid in methanol) at a flow rate of 300 μl per minute. The gradient began at 0% B, reached 35% B by 12 minutes, then increased to 85% B at 14 min and remained at that level until 18 minutes. Subsequently, the mixture returned to 0% B over a period of 3 minutes before a final 6 minute re-equilibration period. Analytes were applied to the mass analyzer using an electrospray ionization probe operating in positive mode with the following condition: capillary voltage, 4000V; fragmentor voltage, 100V, source temperature, 350° C.; nebulizer pressure, 50 PSI; gas flow 10 L/min. Ions were detected in full scan mode where quadrupoles 1 and 2 were set to RF only, whereas the third quadrupole scanned from 100-500 m/z over 525 milliseconds. Substrate acceptance by the enzymes was determined according to the formation of a peak with a 14-m/z increase in mass, corresponding to methylated substrate and a decrease in the substrate peak area, relative to the appropriate boiled control assay.

### Example 2—N-Alkylation of Phentermine

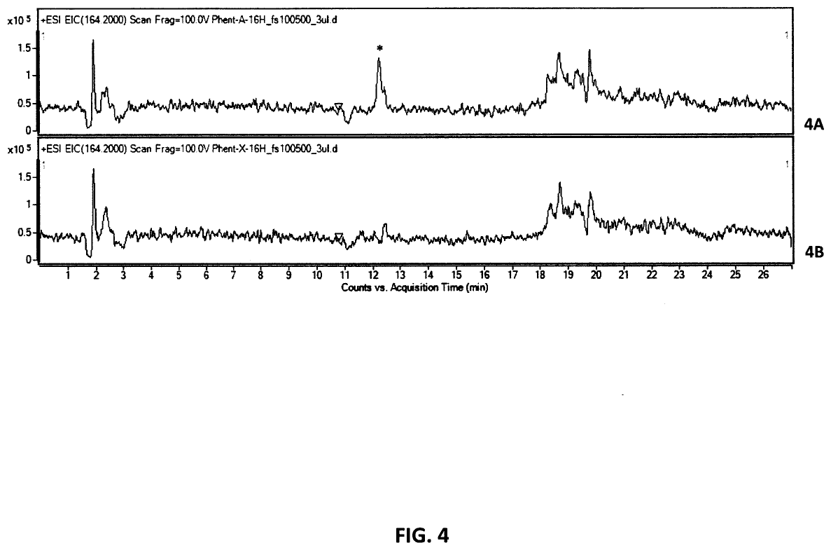

N-alkylation of phentermine using EsNMT was assayed using the methodology described in Example 1. Extracted ion chromatograms 14 m/z greater than the substrate m/z for assays using either native (4A) or heat-denatured (4B) EsNMT enzyme are shown in FIG. 4. Asterisk indicates reaction product.

### Example 3—N-Alkylation of Amphetamine

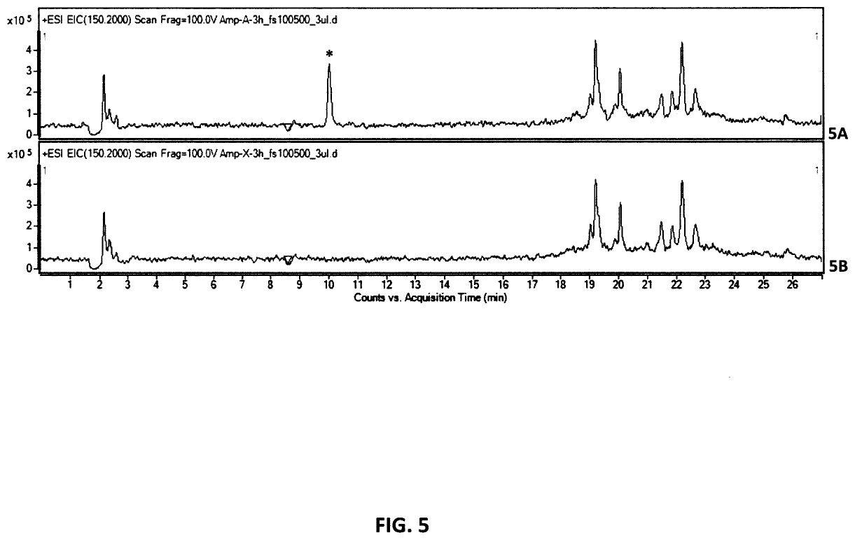

N-alkylation of amphetamine using EsNMT was assayed using the methodology described in Example 1. Extracted ion chromatograms 14 m/z greater than the substrate m/z for assays using either native (5A) or heat-denatured (5B) EsNMT enzyme are shown in FIG. 5. Asterisk indicates reaction product.

### Example 4—N-Alkylation of Cathinone

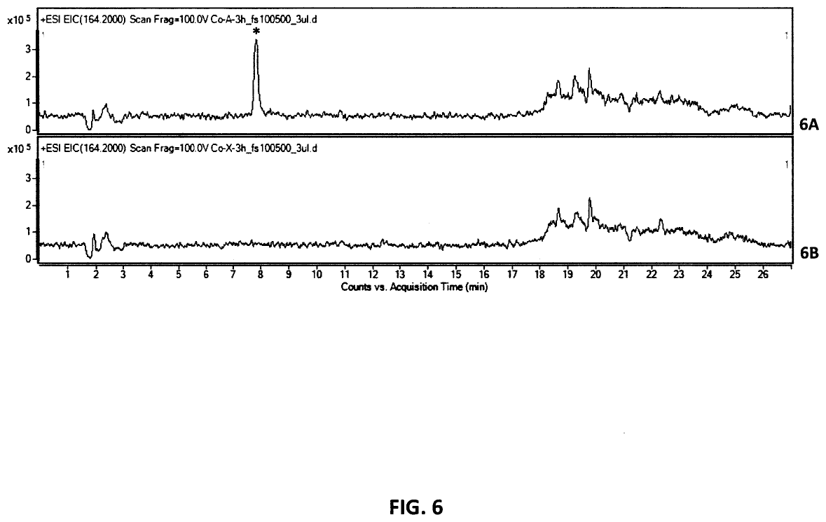

N-alkylation of cathinone using EsNMT was assayed using the methodology described in Example 1. Extracted ion chromatograms 14 m/z greater than the substrate m/z for assays using either native (6A) or heat-denatured (6B) EsNMT enzyme are shown in FIG. 6. Asterisk indicates reaction product.

### Example 5—N-Alkylation of N-Methyl-Cathinone

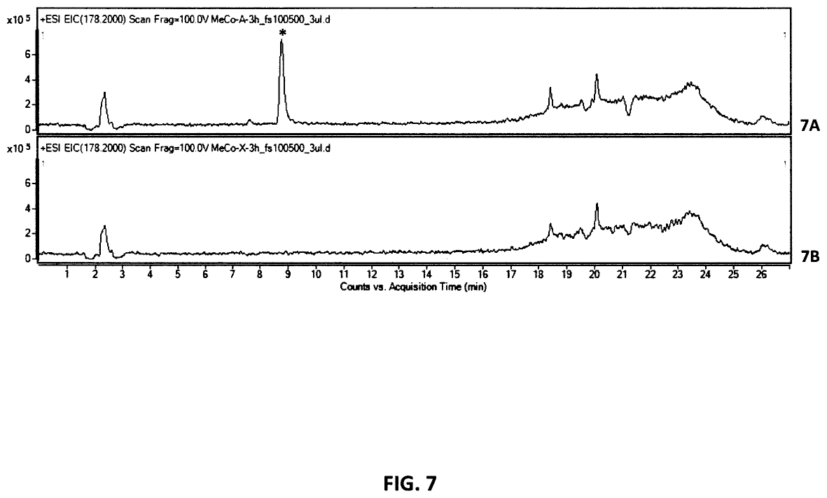

N-alkylation of N-methyl-cathinone using EsNMT was assayed using the methodology described in Example 1. Extracted ion chromatograms 14 m/z greater than the substrate m/z for assays using either native (7A) or heat-denatured (7B) EsNMT enzyme are shown in FIG. 7. Asterisk indicates reaction product.

### Example 6—N-Alkylation of Nor(Pseudo)Ephedrine

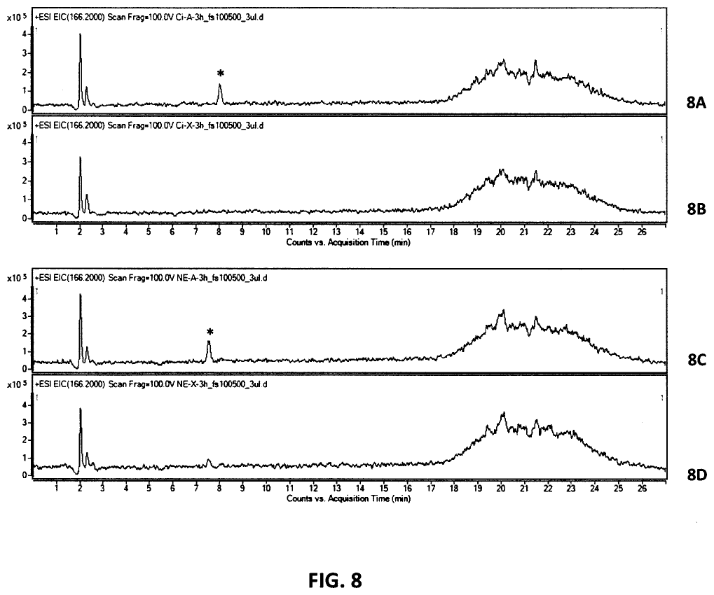

N-alkylation of nor(pseudo)ephedrine, specifically norpseudoephedrine, also known as cathine, and norephedrine, using EsNMT were assayed using the methodology described in Example 1. Extracted ion chromatograms 14 m/z greater than the substrate m/z for assays using either native (8A, 8C) or heat-denatured (8B, 8D) EsNMT enzyme are shown in FIGS. 8A and 8B (norpseudoephedrine), and FIGS. 8C and 8D (norephedrine). Asterisk indicates reaction product.

### Example 7—N-Alkylation of (Pseudo)Ephedrine

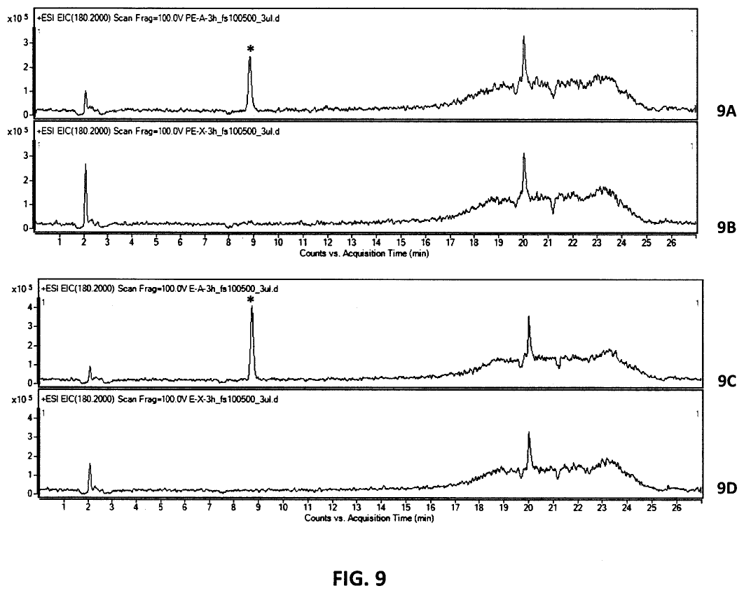

N-alkylation of (pseudo)ephedrine, specifically pseudoephedrine and ephedrine, using EsNMT were assayed using the methodology described in Example 1. Extracted ion chromatograms 14 m/z greater than the substrate m/z for assays using either native (9A, 9C) or heat-denatured (9B, 9D) EsNMT enzyme are shown in FIGS. 9A and 9B (pseudoephedrine), and 9C and 9D (ephedrine). Asterisk indicates reaction product.

### Example 8—N-Alkylation of Tyramine

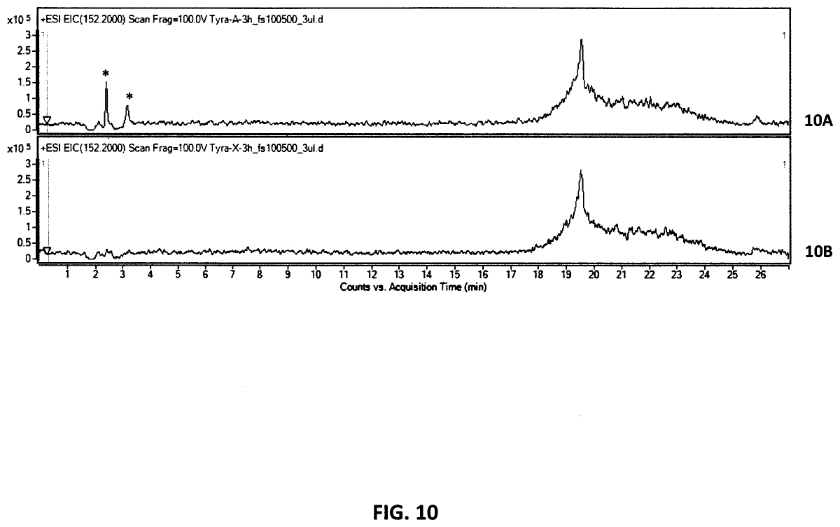

N-alkylation of tyramine using EsNMT was assayed using the methodology described in Example 1. Extracted ion chromatograms 14 m/z greater than the substrate m/z for assays using either native (10A) or heat-denatured (10B) EsNMT enzyme are shown in FIG. 10. Asterisk indicates reaction product.

### Example 9—N-Alkylation of Mescaline

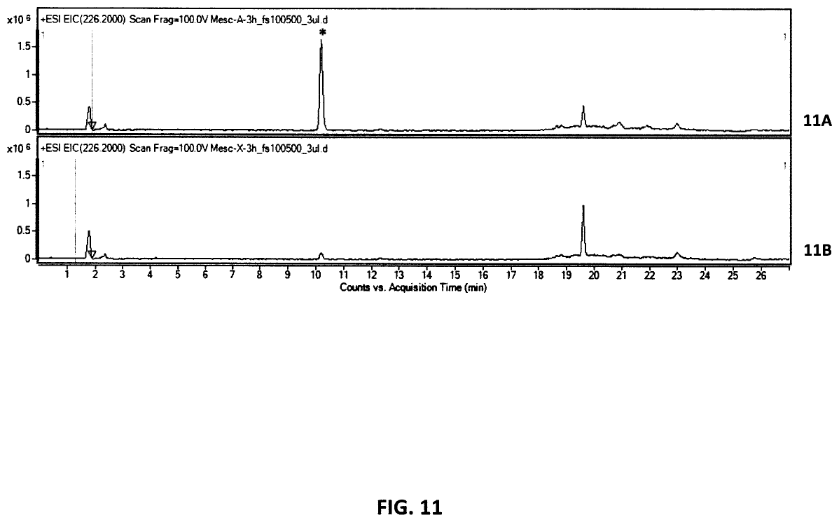

N-alkylation of mescaline using EsNMT was assayed using the methodology described in Example 1. Extracted ion chromatograms 14 m/z greater than the substrate m/z for assays using either native (11A) or heat-denatured (11B) EsNMT enzyme are shown in FIG. 11. Asterisk indicates reaction product.

### Example 10—N-Alkylation of Methylenedioxyamphetamine

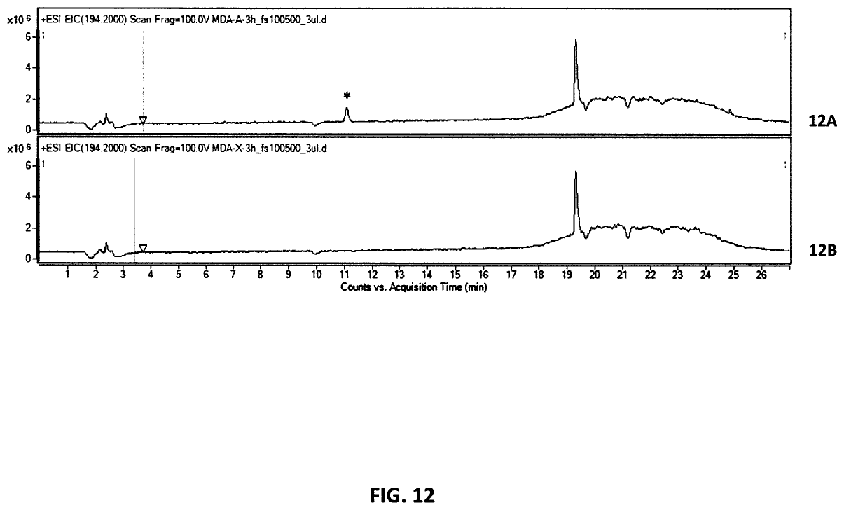

N-alkylation of methylenedioxyamphetamine using EsNMT was assayed using the methodology described in Example 1. Extracted ion chromatograms 14 m/z greater than the substrate m/z for assays using either native (12A) or heat-denatured (12B) EsNMT enzyme are shown in FIG. 12. Asterisk indicates reaction product.

### Example 11—N-Alkylation of Synephrine

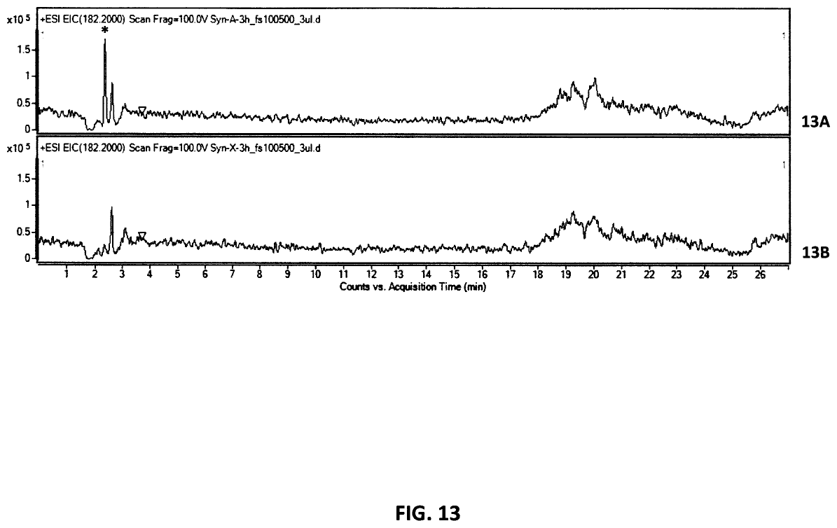

N-alkylation of synephrine using EsNMT was assayed using the methodology described in Example 1. Extracted ion chromatograms 14 m/z greater than the substrate m/z for assays using either native (13A) or heat-denatured (13B) EsNMT enzyme are shown in FIG. 13. Asterisk indicates reaction product.

### Example 12—N-Alkylation of THQ2

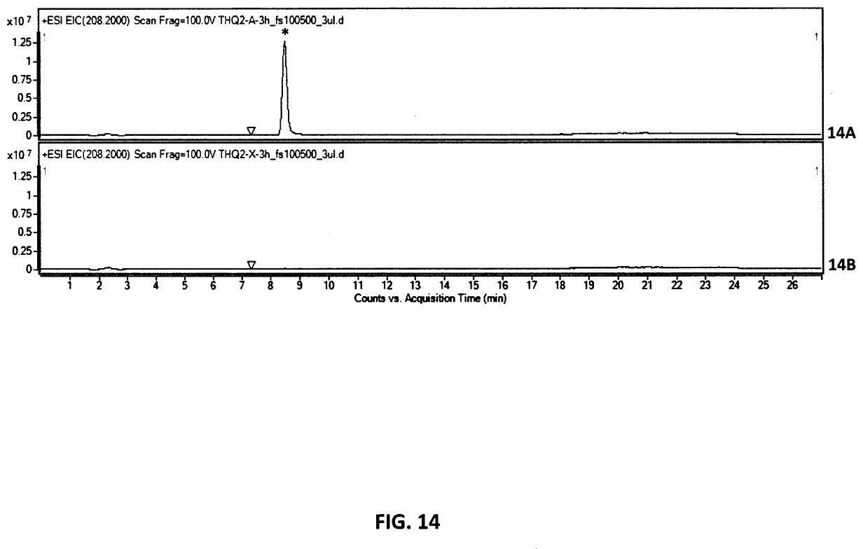

N-alkylation of THQ2 using EsNMT was assayed using the methodology described in Example 1. Extracted ion chromatograms 14 m/z greater than the substrate m/z for assays using either native (14A) or heat-denatured (14B) EsNMT enzyme are shown in FIG. 14. Asterisk indicates reaction product.

### Example 13—N-Alkylation of Reticuline

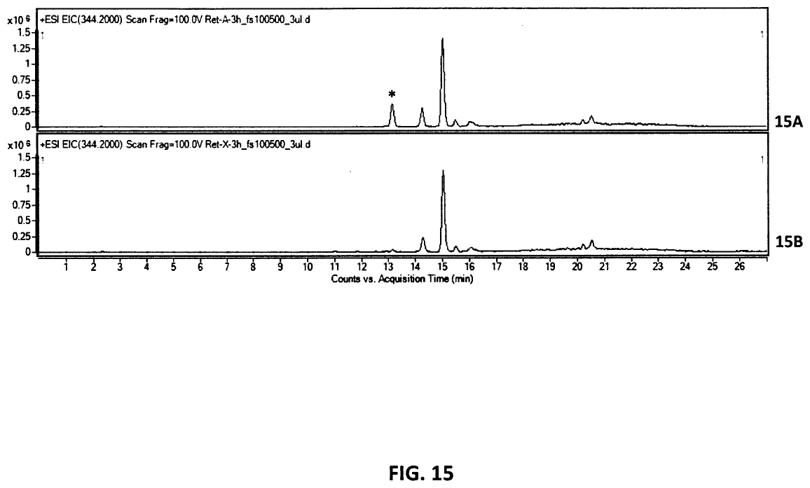

N-alkylation of reticuline using EsNMT was assayed using the methodology described in Example 1. Extracted ion chromatograms 14 m/z greater than the substrate m/z for assays using either native (15A) or heat-denatured (15B) EsNMT enzyme are shown in FIG. 15. Asterisk indicates reaction product.

### Example 14—N-Alkylation of Coclaurine

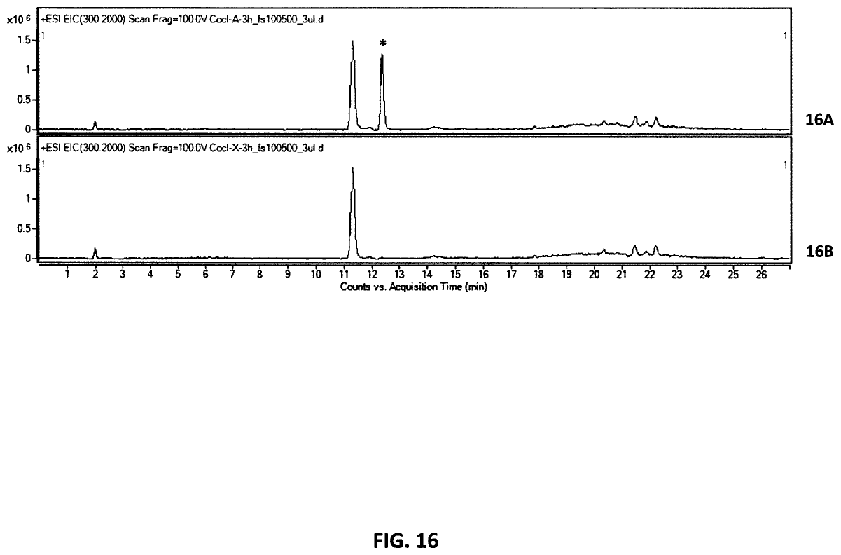

N-alkylation of coclaurine using EsNMT was assayed using the methodology described in Example 1. Extracted ion chromatograms 14 m/z greater than the substrate m/z for assays using either native (16A) or heat-denatured (16B) EsNMT enzyme are shown in FIG. 16. Asterisk indicates reaction product.

### Example 15—N-Alkylation of THQ1

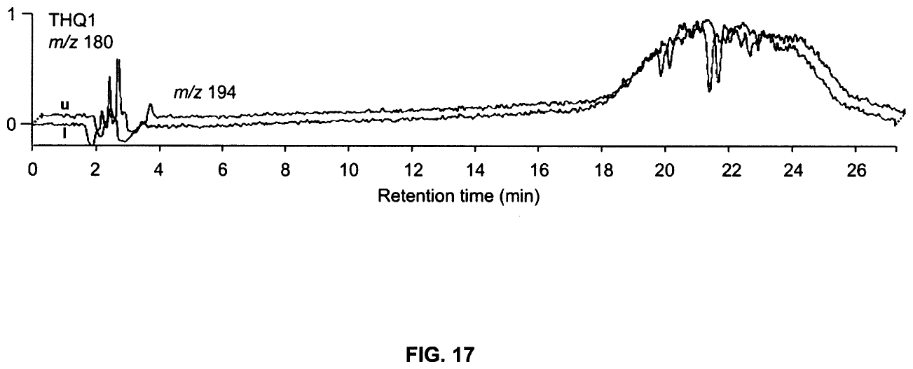

N-alkylation of THQ1 using EsNMT was assayed using the methodology described in Example 1. A pair of extracted ion chromatograms 14 m/z greater than the substrate m/z for assays using either native (upper chromatogram (u)) or heat-denatured EsNMT enzyme (lower chromatogram (1)) is shown in FIG. 17. The highlighted area indicates reaction product.

### Example 16—N-Alkylation of Papaverine

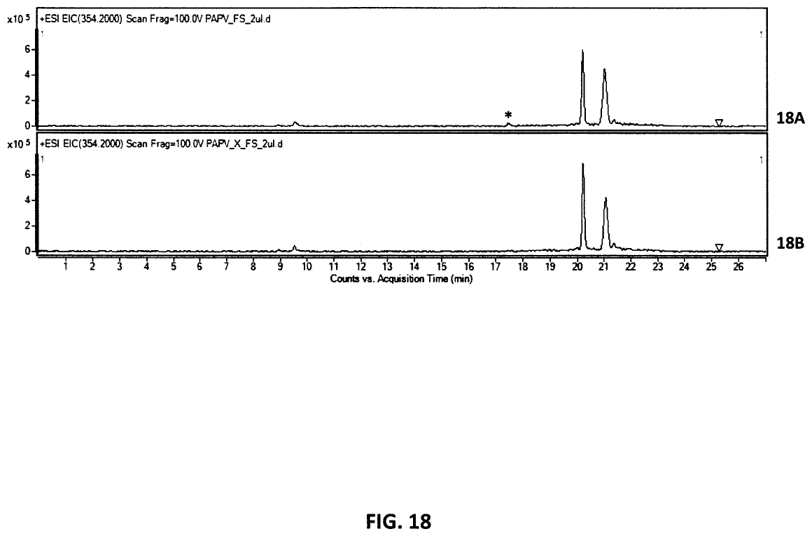

N-alkylation of papaverine using EsNMT was assayed using the methodology described in Example 1. Extracted ion chromatograms 14 m/z greater than the substrate m/z for assays using either native (18A) or heat-denatured (18B) EsNMT enzyme are shown in FIG. 18. Asterisk indicates reaction product.

### Example 17—N-Alkylation of Tryptamine

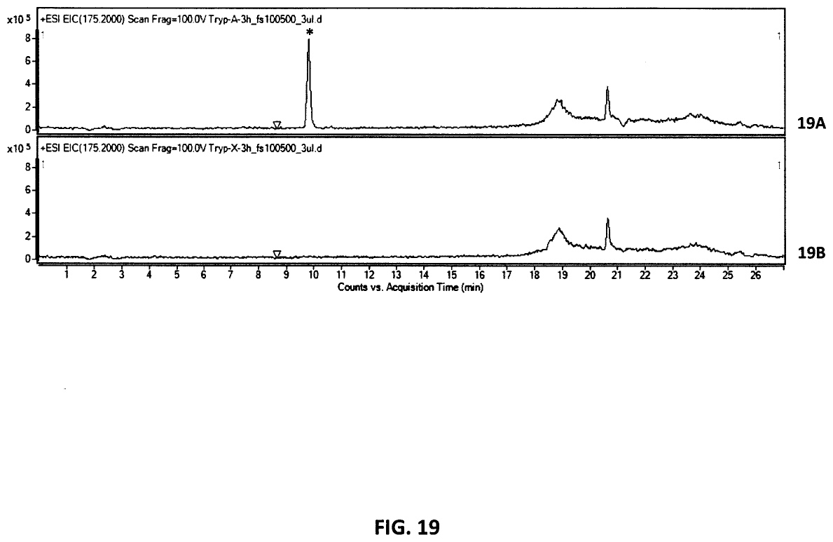

N-alkylation of tryptamine using EsNMT was assayed using the methodology described in Example 1. Extracted ion chromatograms 14 m/z greater than the substrate m/z for assays using either native (19A) or heat-denatured (19B) EsNMT enzyme are shown in FIG. 19. Asterisk indicates reaction products.

### Example 18—N-Alkylation of Harmaline

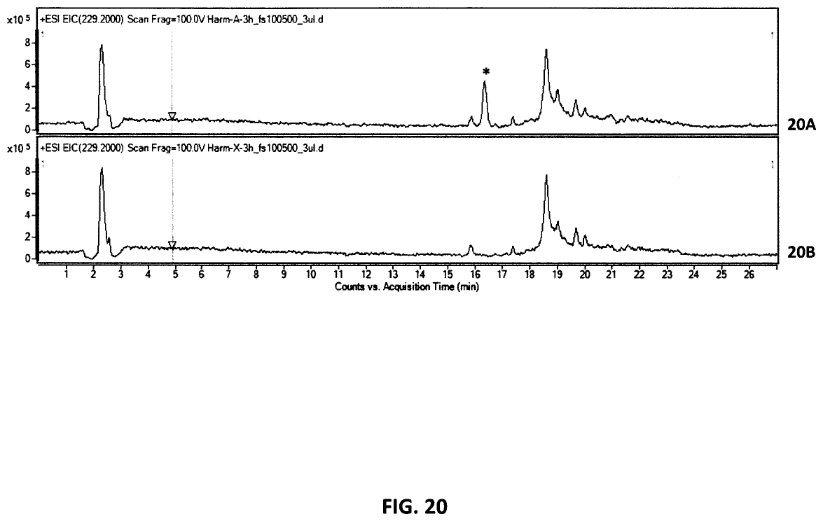

N-alkylation of harmaline using EsNMT was assayed using the methodology described in Example 1. Extracted ion chromatograms 14 m/z greater than the substrate m/z for assays using either native (20A) or heat-denatured (20B) EsNMT enzyme are shown in FIG. 20. Asterisk indicates reaction product.

### Example 19—N-Alkylation of Propanolol

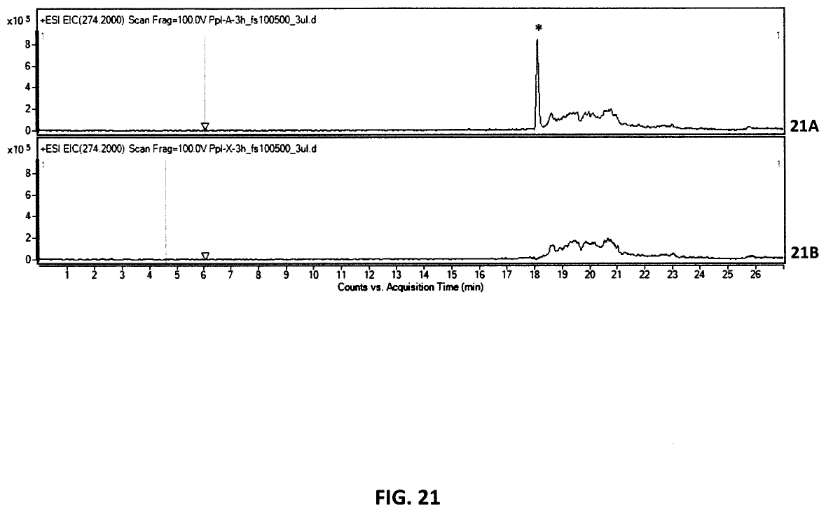

N-alkylation of propanolol using EsNMT was assayed using the methodology described in Example 1. Extracted ion chromatograms 14 m/z greater than the substrate m/z for assays using either native (21A) or heat-denatured (21B) EsNMT enzyme are shown in FIG. 21 Asterisk indicates reaction product.

### Example 20—N-Alkylation of Stylopine

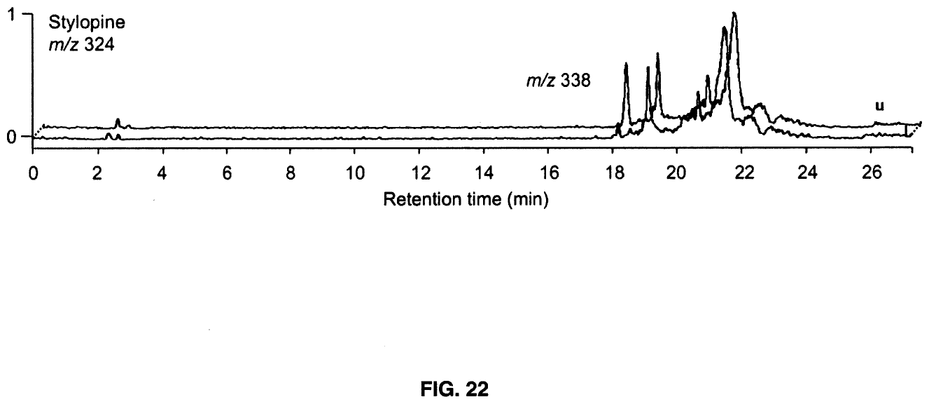

N-alkylation of stylopine using EsNMT was assayed using the methodology described in Example 1. A pair of extracted ion chromatograms 14 m/z greater than the substrate m/z for assays using either native (upper chromatogram (u)) or heat-denatured EsNMT enzyme (lower chromatogram (1)) is shown in FIG. 22. The highlighted area indicates reaction product.

### Example 21—N-Alkylation of Methylephedrine

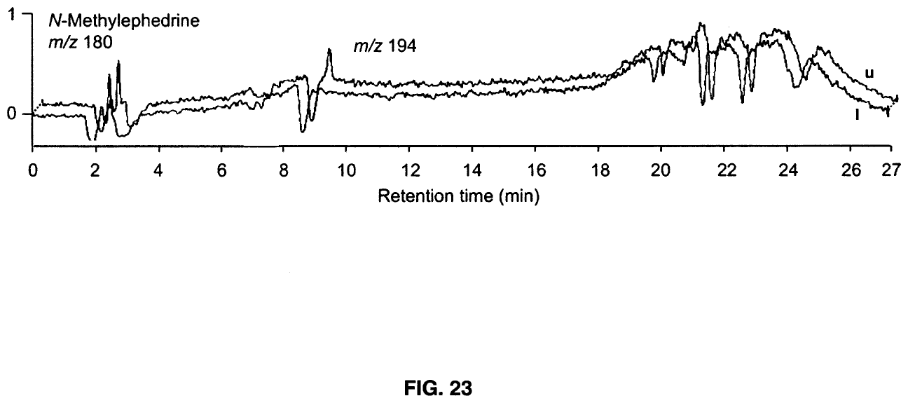

N-alkylation of methylephedrine using EsNMT was assayed using the methodology described in Example 1. A pair of extracted ion chromatograms 14 m/z greater than the substrate m/z for assays using either native (upper chromatogram (u)) or heat-denatured EsNMT enzyme (lower chromatogram (1)) is shown in FIG. 23. The highlighted area indicates reaction product.

### Example 22—Lack of Production of N-Alkylated Substrates (1)

The following alkaloid substrate compounds were incubated with EsNMT (SEQ. ID NO: 2), in accordance with Example 1: (i) taurine; (ii) benzylamine; (iii), 1-phenylethan-1-amine; (iv) octopamine; (v) dopamine; (vi) norlaudanosoline; (vii) noscapine; (viii) harmine; (ix) mitragynine, (x) histamine; (xi) nicotinamide; (xii) anthranilic acid; (xiii) p-dimethylaminobenzaldehyde; (xiv) tropinone; (xv) theobromine; (xvi) xanthosine (xvii) tyrosine; (xviii) 3,4-dihydroxyphenylalanine; (xix) phenylalanine; (xx) tryptophan, and (xxi) adenine. No N-alkylated alkaloid product compounds were detected using any of these alkaloid substrate compounds.

### Example 23—Lack of Production of N-Alkylated Substrates (2)

Each of the alkaloid substrates shown in Table 1 were incubated with the following N-alkyltransferases: GfCNMT (SEQ. ID NO: 3), gfTNMT (SEQ. ID NO: 4) and Ps RNMT (SEQ. ID NO: 4) and EsNMT (SEQ. ID NO: 2), as described in Example 1. The presence of N-alkylated alkaloid product was detected as described in Examples 1-20. The results obtained are provided in Table 1. Legend: ND=Not Determined; −=no detectable quantities of product N-alkylated compound; and Trace, +, ++, +++=increasing relative levels of product N-alkylated compound.

TABLE 1
N-alkylation of substrate alkaloid compounds

| | Enzyme | Enzyme | Enzyme | Enzyme |
|---|---|---|---|---|
| Substrate | GfCNMT | GfTNMT | PdRNMT | EsNMT |
| Taurine | ND | ND | ND | − |
| Benzylamine | ND | ND | ND | − |
| 1-Phenylethaneamine | ND | ND | ND | − |
| 2-Phenylethaneamine | Trace | Trace | − | + |
| Phentermine | ND | ND | ND | + |
| Amphetamine | Trace | Trace | − | + |
| Cathinone | ND | ND | ND | + |
| N-Methyl-cathinone | ND | ND | ND | + |
| Norephedrine | − | − | − | + |
| Norpseudoephedrine | ND | ND | ND | + |
| Ephedrine | − | ? | − | + |
| Pseudoephedrine | ND | ND | ND | + |
| Methylephedrine | ND | ND | ND | + |
| Tyramine | + | − | − | + |
| Mescaline | − | − | − | + |
| Methylenedioxyamphetamine | − | − | − | + |
| Octopamine | ND | ND | ND | − |
| Synephrine | ND | ND | ND | + |
| Dopamine | ND | ND | ND | − |
| THQ1 | ND | ND | ND | + |
| THQ2 | +++ | + | + | +++ |
| Norlaudanosoline | ND | ND | ND | − |
| S-Reticuline | + | ++ | +++ | + |
| Coclaurine | +++ | + | ++ | + |
| Papaverine | ND | ND | ND | Trace |
| Stylopine | + | + | +++ | + |
| Noscapine | Trace | +++ | ++ | − |
| Tryptamine | Trace | Trace | Trace | +++ |
| Harmaline | + | − | − | + |
| Harmine | ++ | − | − | − |
| Mitragynine | ND | ND | ND | − |
| Propanolol | ++ | +++ | +++ | + |
| Histamine | − | − | − | − |
| Nicotinamide | − | − | − | − |
| Anthrallic acid | ND | ND | ND | − |
| p-dimethylaminobenzaldehyde | ND | ND | ND | − |
| Tropinone | ND | ND | ND | − |
| Theobromine | ND | ND | ND | − |
| Xanthosine | ND | ND | ND | − |
| Tyrosine | ND | ND | ND | − |
| 3,4-dihydroxyphenylalanine | ND | ND | ND | − |
| Phenylalanine | ND | ND | ND | − |
| Tryptophan | ND | ND | ND | − |
| Adenine | ND | ND | ND | − |

1: A method of making an alkaloid comprising:
(a) providing an alkaloid substrate selected from the group of substrates consisting of:
(i) a first alkaloid compound (I): 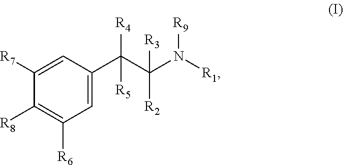
wherein
R₁ is a hydrogen atom or an alkyl group;
R₂ and R₃ are independently or simultaneously a hydrogen atom, a halogen or an alkyl group;
R₄ is a hydrogen atom, a hydroxy group, a halogen, or an alkyl group;
R₅ is a hydrogen atom, a halogen, or an alkyl group;
or R₄ and R₅, taken together, form a carbonyl group;
R₆ is a hydrogen atom, a halogen, an alkyl group or an alkoxy group;
R₇ is a hydrogen atom, a halogen, an alkyl group or an alkoxy group;
R₈ is a hydrogen atom, a hydroxy group, a halogen, an alkyl group or an alkoxy group;
R₇ and R₈, taken together, form a methylenedioxy group; and
R₉ is a hydrogen atom or an alkyl group;
(ii) a second alkaloid compound (II): 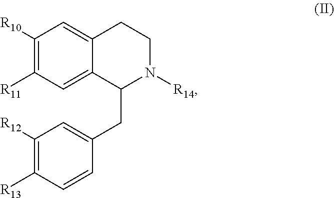
wherein
R₁₀ is a hydrogen atom, a hydroxy group, a halogen, an alkyl group or an alkoxy group;
R₁₁ is a hydrogen atom, a hydroxy group, a halogen, an alkyl group or an alkoxy group;
R₁₂ is a hydrogen atom, a hydroxy group, a halogen, an alkyl group or an alkoxy group;
R₁₃ is a hydrogen atom, a hydroxy group, a halogen, an alkyl group or an alkoxy group; and
wherein R₁₀-R₁₃ are not each simultaneously a hydroxy group; and
R₁₄ is a hydrogen atom or an alkyl group;
(iii) a third alkaloid compound (Ill): 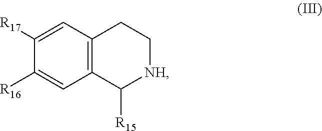
wherein
R₁₅, R₁₆ and R₁₇ are independently or simultaneously a hydrogen atom, a hydroxy group, an alkoxy group, a halogen or an alkyl group; and
(iv) a fourth alkaloid compound (IV) selected from stylopine; tryptamine; harmaline; and propanolol; and
(b) contacting the alkaloid substrate with sufficient quantities of an alkyl donor compound and catalytic quantities of an enzyme encoded by a nucleic acid sequence selected from the group consisting of:
(i) SEQ. ID NO: 1 or SEQ. ID NO: 6;
(ii) a nucleic acid sequence that is substantially identical to SEQ. ID NO: 1 or SEQ. ID NO: 6;
(iii) a nucleic acid sequence that is substantially identical to SEQ. ID NO: 1 or SEQ. ID NO: 6 but for the degeneration of the genetic code;
(iv) a nucleic acid sequence that is complementary to SEQ. ID NO: 1 or SEQ. ID NO: 6;
(v) a nucleic acid sequence encoding a polypeptide having the amino acid sequence set forth in SEQ. ID NO: 2 or SEQ. ID NO: 7;
(vi) a nucleic acid sequence that encodes a functional variant of a polypeptide having the amino acid sequence set forth in SEQ. ID NO: 2 or SEQ. ID NO: 7; and
(vii) a nucleic acid sequence that hybridizes under stringent conditions to any one of the nucleic acid sequences set forth in (i), (ii), (iii), (iv), (v) or (vi),
under reaction conditions permitting an enzyme-catalyzed N-alkylation of the alkaloid substrate to form a N-alkylated alkaloid product.

2: The method according to claim 1, wherein the alkyl donor compound is a methyl donor compound and the enzyme is a methyltransferase capable of N-methylation of the alkaloid substrate to form a N-methylated alkaloid product.

3-38. (canceled)

39. The method according to claim 1, wherein in compound (I):
R₁ is a hydrogen atom, or a methyl group;
R₂ is a hydrogen atom, or a methyl group;
R₃ is a hydrogen atom, or a methyl group;
R₄ is a hydrogen atom, or a hydroxy group; and R₅ is a hydrogen atom; or R₄ and R₅, taken together, are a carbonyl group;
R₆ is a hydrogen atom, or a methoxy group;
R₇ is a hydrogen atom, or a methoxy group;
R₈ is a hydrogen atom, a hydroxy group, or a methoxy group;
R₇ and R₈, taken together, form a methylenedioxy group; and
R₉ is a hydrogen atom, or a methyl group.

40. The method according to claim 1, wherein in compound (II):
R₁₀ is a hydroxy group or a methoxy group;
Ru is a hydroxy group or a methoxy group;
R₁₂ is a hydrogen atom, a hydroxy group, or a methoxy group;
R₁₃ is a hydroxy group or a methoxy group; and
R₁₄ is a hydrogen atom or a methyl group.

41: The method according to claim 1, wherein in compound (Ill):
R₁₅ is a hydrogen atom or a methyl group;
R₁₆ is a hydroxy group or a methoxy group; and
R₁₇ is a hydroxy group, or a methoxy group;

42: The method according to claim 1, wherein the alkaloid substrate is selected from the group consisting of 2-phenylethan-1-amine, phentermine, amphetamine, cathinone, N-methyl-cathinone, nor(pseudo)ephedrine, (pseudo)ephedrine, methyl-(pseudo)ephedrine, tyramine, mescaline, methylenedioxyamphetamine, synephrine, THQ1, THQ2, reticuline, coclaurine, papaverine, stylopine, tryptamine, harmaline and propranolol.

43-62. (canceled)

63: The method according to claim 1, wherein the alkaloid substrate comprises a primary amine and the enzyme-catalyzed N-alkylation forms an N-alkylated alkaloid product comprising a secondary amine.

64: The method according to claim 1, wherein the alkaloid substrate comprises a secondary amine and the enzyme-catalyzed N-alkylation forms an N-alkylated alkaloid product comprising a tertiary amine.

65: The method according to claim 1, wherein the alkaloid substrate comprises a tertiary amine and the enzyme-catalyzed N-alkylation forms an N-alkylated alkaloid product comprising a quaternary amine.

66. (canceled)

67: The method according to claim 1, wherein the reaction conditions are in vitro reaction conditions.

68: The method according to claim 1, wherein the reaction conditions can be are in vivo reaction conditions.

69: A method for preparing an N-alkylated product alkaloid compound according to claim 1 comprising:
(A) providing a chimeric nucleic acid sequence comprising as operably linked components:
(a) a nucleic acid sequence encoding an N-alkyltransferase polypeptide comprising a polypeptide sequence encoded by a nucleic acid sequence selected from the group consisting of:
(i) SEQ. ID NO: 1 or SEQ. ID NO: 6;
(ii) a nucleic acid sequence that is substantially identical to SEQ. ID NO: 1 or SEQ. ID NO: 6;
(iii) a nucleic acid sequence that is substantially identical to SEQ. ID NO: 1 or SEQ. ID NO: 6 but for the degeneration of the genetic code;
(iv) a nucleic acid sequence that is complementary to SEQ. ID NO: 1 or SEQ. ID NO: 6;
(v) a nucleic acid sequence encoding a polypeptide having the amino acid sequence set forth in SEQ. ID NO: 2 or SEQ. ID NO: 7;
(vi) a nucleic acid sequence that encodes a functional variant of a polypeptide having the amino acid sequence set forth in SEQ. ID NO: 2 or SEQ. ID NO: 7; and
(vii) a nucleic acid sequence that hybridizes under stringent conditions to any one of the nucleic acid sequences set forth in (i), (ii), (iii), (iv), (v) or (vi); and
(b) one or more nucleic acid sequences capable of controlling expression in a host cell; and
(B) introducing the chimeric nucleic acid sequence into a host cell capable of producing a substrate alkaloid compound having chemical formula (I), (II), (Ill), or (IV), and growing the host cell to produce the N-alkyltransferase to N-methylate the substrate alkaloid compound and produce a N-alkylated product alkaloid compound.

70: The method according to claim 69, comprising a step (c) comprising recovering the N-alkylated product alkaloid compound.

71: A substantially pure nucleic acid comprising one or more nucleic acid sequences selected from the group consisting of:
(i) SEQ. ID NO: 1 or SEQ. ID NO: 6;
(ii) a nucleic acid sequence that is substantially identical to SEQ. ID NO: 1 or SEQ. ID NO: 6;
(iii) a nucleic acid sequence that is substantially identical to SEQ. ID NO: 1 or SEQ. ID NO: 6 but for the degeneration of the genetic code;
(iv) a nucleic acid sequence that is complementary to SEQ. ID NO: 1 or SEQ. ID NO: 6;
(v) a nucleic acid sequence encoding a polypeptide having the amino acid sequence set forth in SEQ. ID NO: 2 or SEQ. ID NO: 7;
(vi) a nucleic acid sequence that encodes a functional variant of a polypeptide having the amino acid sequence set forth in SEQ. ID NO: 2 or SEQ. ID NO: 7; and
(vii) a nucleic acid sequence that hybridizes under stringent conditions to any one of the nucleic acid sequences set forth in (i), (ii), (iii), (iv), (v) or (vi).

72: A substantially pure polypeptide encoded by the nucleic acid of claim 71.

73: A chimeric nucleic acid sequence comprising as operably linked components:
(a) a nucleic acid sequence of claim 71; and
(b) a nucleic acid sequence capable of controlling expression of the nucleic acid sequence in a host cell.

74: A recombinant expression vector comprising as operably linked components:
(a) a nucleic acid sequence capable of controlling expression in a host cell; and
(b) a nucleic acid sequence of claim 71

75: A host cell comprising a recombinant nucleic acid sequence of claim 71.

76-80. (canceled)

81: The method according to claim 69 wherein the host cell is a microbial cell.

82: The method according to claim 81 wherein the microbial cell is a yeast cell or a bacterial cell.
